(19) United States
(12) Patent Application Publication
(10) Pub. No.: US 2024/0141295 A1
(43) Pub. Date: May 2, 2024

(54) NOVEL CELL LINES, METHODS OF PRODUCING NATURAL KILLER CELLS AND USES THEREOF

(71) Applicant: City of Hope, Duarte, CA (US)

(72) Inventors: Jianhua Yu, Duarte, CA (US); Michael A. Caligiuri, Pasadena, CA (US); Ting Lu, Duarte, CA (US)

(21) Appl. No.: 18/023,536

(22) PCT Filed: Aug. 31, 2021
(86) PCT No.: PCT/US2021/048539

§ 371 Date: Feb. 27, 2023

Related U.S. Application Data
(60) Provisional application No. 63/219,760, filed on Jul. 8, 2021.
(60) Provisional application No. 63/072,657, filed on Aug. 31, 2020.

(51) Int. Cl. C12N 5/0783; A61K 39/00; A61P 35/00; C07K 14/54; C07K 14/705; C07K 14/725; C12N 9/12

(52) U.S. Cl. CPC C12N 5/0646; A61K 39/4613; A61K 39/4631; A61K 39/464499; A61P 35/00; C07K 14/54; C07K 14/5443; C07K 14/7051; C07K 14/70578; C12N 9/12; C12Y 207/10001; C07K 2317/24; C12N 2501/2302; C12N 2502/11

(57) ABSTRACT

This disclosure features novel cell lines and related methods for producing human natural killer (NK) cells, compositions of the NK cells produced by these methods, and uses thereof.

### CROSS-REFERENCE TO RELATED APPLICATIONS

This application claims priority and benefit from U.S. Provisional Patent Application 63/072,657, filed Aug. 31, 2020; and U.S. Provisional Patent Application 63/219,760 filed Jul. 8, 2021, the contents and disclosures of which are incorporated herein by reference in their entireties.

### REFERENCE TO SEQUENCE LISTING

This instant application contains a Sequence Listing which has been submitted electronically in ASCII format and is hereby incorporated by reference in its entirety. Said ASCII copy, created on Aug. 24, 2021, is named 40056-0065WO1_SL.txt and is 43,592 bytes in size.

### TECHNICAL FIELD

Novel cell lines and related methods for producing human natural killer (NK) cells, in particular NK cells with improved expansion efficiency and enhanced cell function, and methods of using the expanded NK cells for anti-cancer and anti-viral treatment. More particularly, the application relates to the ex vivo, or in vitro co-culture of NK cells with novel Tyro3⁺ feeder cells, thereby facilitating production of an expanded NK cell population.

### BACKGROUND

Cancer is the second leading cause of death globally, accounting for an estimated 9.6 million deaths, or one in six deaths, in 2018. Natural killer (NK) cell-based immunotherapy is a promising therapeutic approach for cancerous tumors and hematological malignancies. NK cells exert strong cytotoxic effects when they encounter cells lacking self-MHC class I molecules. Thus, NK cells are able to recognize and eliminate tumor cells, which may downregulate MHC class I molecules, making them ideal candidates for tumor immunotherapy. Similarly, NK cells also mediate immune responses to virally infected cells, which downregulate MHC class I expression. Thus, NK cell immunotherapy a promising tool for antiviral therapy.

In spite of the advantageous properties of NK cells in killing tumor cells and virally infected cells, they remain difficult to work with and to apply in immunotherapy, primarily due to the difficulty in obtaining sufficient numbers of activated NK cells for adoptive transfer. Further, it may be challenging to maintain their tumor-targeting and tumoricidal capabilities during culture and expansion. Thus, there is a need for improved in vitro and ex vivo methods for expanding NK cell populations to produce high numbers of NK cells with improved functions, such that they may be effective in targeting and eliminating tumor cells and virally infected cells when used in vivo.

### SUMMARY

Described herein are novel cell lines and related methods for producing an expanded population of NK cells by using feeder cells expressing Tyro3 (e.g., Tyro3⁺ K562 cells) and compositions comprising the expanded population of NK cells produced by such methods. Also described herein are methods for treating cancer and viral infections using compositions of the disclosure.

This disclosure is based on the surprising observation that when human NK cells (which do not express endogenous Tyro3 on their cell surface) come into contact with K562 tumor cells overexpressing Tyro3 (Tyro3⁺ K562 cells) in culture, the NK cells can acquire Tyro3 from the surface of the K562 cells via trogocytosis and display the acquired Tyro3 on NK cell surfaces. The Tyro3⁺ NK cells thus produced have a better expansion efficiency, and significantly enhanced cytotoxicity, IFN-γ production and activated surface markers expression compared to Tyro3⁻ NK cells.

Described herein are modified K562 myeloid leukemia cells, wherein the modified K562 cells (Tyro3⁺ K562 cells) exogenously express human Tyro3 polypeptide. The Tyro3 polypeptide comprises an amino acid sequence that is at least 95% identical to SEQ ID NO:2. For instance, the Tyro3 polypeptide comprises an amino acid sequence that is at least 95%, at least 96%, at least 97%, at least 98%, or at least 99% identical to SEQ ID NO:2. The Tyro3⁺ K562 cells express membrane bound interleukin 21 (IL-21) and 4-1 BB ligand (4-1BBL).

In some embodiments, the Tyro3⁺ K562 cells may enhance the expansion of human natural killer (NK) cells that contact the K562 cells by at least about 10% compared to K562 cells that do not express exogenous human Tyro3 (Tyro3⁻ K562 cells). The Tyro3⁺ K562 cells may enhance the expansion of human NK cells that contact the K562 cells by at least about 10%, 15%, 20%, 25%, or greater than 30% compared to the expansion of human NK cells that contact Tyro3⁻ K562 cells. In various embodiments, the human Tyro3 expression in the K562 cells is under the control of a strong promoter. In some embodiments, the strong promoter is a constitutive promoter. In some embodiments, the strong promoter is a retroviral promoter.

Also described herein is method of expanding a population of human NK cells, comprising co-culturing the population of NK cells with modified K562 myeloid leukemia cells. The modified K562 cells (Tyro3⁺ K562 cells) exogenously express human Tyro3 polypeptide. The Tyro3 polypeptide comprises an amino acid sequence that is at least 95% identical to SEQ ID NO:2. For instance, the Tyro3 polypeptide comprises an amino acid sequence that is at least 95%, at least 96%, at least 97%, at least 98%, or at least 99% identical to SEQ ID NO:2. The Tyro3⁺ K562 cell expresses membrane bound interleukin 21 (IL-21) and 4-1 BB ligand (4-1BBL). In various embodiments, the human Tyro3 expression in the K562 cells is under the control of a strong promoter. In some embodiments, the strong promoter is a constitutive promoter. In some embodiments, the strong promoter is a retroviral promoter.

In various embodiments of the above methods, the Tyro3 is transferred from the Tyro3⁺ K562 cells to the human NK cells by trogocytosis to obtain Tyro3⁺ NK cells. This can be accomplished, e.g., by co-culturing Tyro3⁺ K562 cells and NK cell. In some embodiments, the human NK cells are expanded in the presence of interleukin 2 (IL-2). In some embodiments, the ratio of human NK cells to Tyro3⁺ K562 cells may be in a range of about 0.1:1 to about 10:1. For instance, the ratio of human NK cells to K562 cells may be about 0.1:1, 0.3:1, 0.4:1, 0.7:1, 0.9:1, 1:1, 2:1, 3:1, 4:1, 5:1, 6:1, 7:1 8:1, 9:1, 10:1, or greater than 10:1.

In some embodiments of the above methods, the NK cells and the Tyro3⁺ K562 cells cells are co-cultured for a duration of about 5 min to about 6 weeks. For instance, the NK cells and the Tyro3⁺ K562 cells cells may be co-cultured for about 5 min, 30 min, 1 hour, 2 hours, 12 hours, 24 hours, 1 week, 2 weeks, 3 weeks, 4 weeks, 5 weeks, 6 weeks, or greater than 6 weeks.

In some embodiments of the above methods, the IL-2 is present at a concentration of about 0 IU/ml to about 5000 IU/ml. In some embodiments, the IL-2 is present at a concentration of about 50 IU/ml to about 2000 IU/ml. In other embodiments, the IL-2 is present at a concentration of about 150 IU/ml to about 900 IU/ml.

In some embodiments of the above methods, the expanded NK cells are activated NK cells. In some embodiments, the NK cells are engineered NK cells. In some embodiments, the engineered NK cells express a chimeric antigen receptor (CAR).

In some embodiments of the above methods, the expanded NK cells are Programmed death-ligand 1 (PD-L1)⁺ cells. In some embodiments, the expanded NK cells have at least one of: (a) higher levels of mRNA and/or protein of one or more of the following markers: phosphorylated-signal transducer and activator of transcription 3 (p-STAT3), phosphorylated-NF-κB p65 (p-P65), pSTAT5, phospho-Akt (p-AKT) and phospho-extracellular signal-related kinase (p-ERK), Interferon-gamma (IFNγ), Cluster of differentiation 107a (CD107a), CD25, CD69, Killer cell lectin-like receptor subfamily G member 1 (KLRG1), when compared to reference levels of the corresponding mRNA and/or protein in a control; and (b) higher levels of activity of one or more of the following markers: p-STAT3, p-P65, pSTAT5, p-AKT, p-ERK, IFNγ, CD107a, CD25, CD69, and KLRG1, when compared to reference levels of the corresponding activity in a control.

In some embodiments, the Tyro3+ K562 cells harbor an exogenous nucleotide sequence encoding human Tyro3.

In some embodiments, the Tyro3+ cells further express one or more of CD64 (FcγRI), CD86 (B7-2), and truncated CD19 (tCD19). In some embodiments, the Tyro3+ K562 cells further express membrane bound IL15, soluble IL15 or an IL15 sushi receptor complex.

Also described herein is a composition comprising the population of expanded NK cells produced by the above methods.

Also described herein is a composition comprising expanded NK cells, wherein at least 20% of the NK cells in the population are Tyro3⁺ NK cells.

Also described herein is method of treating cancer in a subject in need thereof, comprising administering to the subject a therapeutically effective amount of a composition of the disclosure, thereby treating cancer in the subject. In some embodiments, the cancer is selected from a group consisting of lung cancer, breast cancer, ewing sarcoma, central nervous system neoplasm, skin cancer, head and neck cancer, ovarian cancer, colon cancer, anal cancer, stomach cancer, gastrointestinal cancer, fallopian tube cancer, endometrial cancer, cervical cancer, vaginal cancer, vulvar cancer, esophageal cancer, endocrine cancer, thyroid cancer, parathyroid cancer, adrenal cancer, soft tissue sarcoma, urethral cancer, penile cancer, testicular cancer, brain stem glioma, pituitary cancer, adrenocortical cancer, gallbladder cancer, multiple myeloma, cholangiocarcinoma, fibrosarcoma, lymphoma, liver cancer, kidney cancer, bone cancer, bladder cancer, colorectal cancer, endometrial cancer, renal cell cancer, pancreatic cancer, prostate cancer, thyroid cancer, mesothelioma, neuroblastoma, retinoblastoma, melanoma, rhabdomyosarcoma, leukemia and lymphoma.

Also described herein is a method of treating a viral infection in a subject in need thereof, comprising administering to the subject a therapeutically effective amount of a composition of the disclosure, thereby treating the viral infection in the subject. In some embodiments, the viral infection is caused by human immunodeficiency virus (HIV), Epstein-Barr virus (EBV), herpes simplex virus (HSV), cytomegalovirus (CMV), varicella-zoster virus (VZV), hepatitis B virus (HBV) or hepatitis C virus (HCV), and coronavirus.

Also described is a method of suppressing the proliferation of tumor cells comprising contacting the tumor cells with a therapeutically effective amount of a composition of the disclosure. In some embodiments, the tumor cells are primary ductal carcinoma cells, glioblastoma cells, leukemia cells, acute T cell leukemia cells, chronic myeloid lymphoma (CML) cells, acute myelogenous leukemia cells, chronic myelogenous leukemia (CML) cells, lung carcinoma cells, colon adenocarcinoma cells, histiocytic lymphoma cells, multiple myeloma cells, colorectal carcinoma cells, colorectal adenocarcinoma cells, prostate cancer cells, or retinoblastoma cells.

The materials, methods, and examples are illustrative only and not intended to be limiting. All publications, patent applications, patents, sequences, database entries, and other references mentioned herein are incorporated by reference in their entirety for any and all purposes. Other features and advantages of the described compositions and methods will be apparent from the following detailed description and figures, and from the claims.

### DESCRIPTION OF DRAWINGS

FIGS. 1A-E depict expression levels of Tyro3, Axl, and Mertk in CD56⁺ NK cells after co-culture of primary human NK cells with or without K562 cells for 24 hours at an effector (E)/tumor (T) ratio of 10:1. FIG. 1A shows representative flow cytometry plots from 4 different donors. Summary data of FIG. 1A are shown in FIG. 1B. Paired t test was used for two-group comparisons. FIG. 1C depicts Tyro3 expression on NK cells as measured by flow cytometry when primary human NK cells were co-cultured with K562 cells at a 1:1 E/T ratio for the indicated time points. Data are summarized from 4 different donors. FIG. 1D depicts Tyro3 expression on NK cells as determined by flow cytometry when primary human NK cells were pretreated with or without IL-2 (150 IU/ml) overnight, and then co-cultured with K562 cells at a 1:1 E/T ratio. Data are summarized from 7 different donors. FIG. 1E depicts summary data of Tyro3 expression in two subsets of NK cells from FIG. 1D (CD56^{bright} and CD56^{dim} NK subsets). Representative flow cytometry plots and data are summarized from 7 different donors. One-way ANOVA was used for FIGS. 1B, 1D and 1E. P values were adjusted by the Holm-Sidak method. *P<0.05, **P<0.01, ****P<0.0001;. Data are presented as mean±SD.

FIGS. 2A-2D depict expression levels of Tyro3 on NK cells co-cultured under various conditions. FIG. 2A depicts expression levels of Tyro3 expression on NK cells co-cultured with or without K562 cells in transwell plates, or co-cultured directly with or without K562 cells, or co-cultured with or without supernatant from K562 culture for 1 hr at an E/T ratio of 1:1. FIG. 2B depicts representative flow cytometry plots and summary data (n=5) showing Tyro3 expression on NK cells from IL-2 stimulated NK cells co-cultured directly with or without K562, or with or without supernatant from NK cells and K562 co-culture for 1 hr at an E/T ratio of 1:1. FIG. 2C depicts representative flow cytometry plots and summary data (n=4) showing Tyro3 expression on NK cells from IL-2 stimulated NK cells co-cultured with K562 or K562^{Tyro3-KO} cells for 1 hr at an E/T ratio of 1:1. FIG. 2D depicts representative flow cytometry plots and summary data showing the Tyro3 expression on NK cells from IL-2-stimulated NK cells co-cultured with Molm-13 or Molm-13^{Tyro3-OE} cells for 1 hr at an E/T ratio of 1:1. FIG. 2E depicts nonlinear regression analysis of the correlation between the percentages of acquired Tyro3 on NK cells and their corresponding Tyro3% on K562 cells. The correlation as calculated by Pearson test was statistically significant. Data are summarized from 3 different donors. FIGS. 2F-2G depicts summary of kinetics of persistence of acquired Tyro3 on NK cells (after sorting separation) from 4 independent experiments for NK cells pretreated with or without IL-2 (150 IU/ml) and co-cultured with K562 cells for 1 hr at an E/T ratio of 1:1. FIG. 2F shows relative % Tyro3⁺ NK cells in the presence or absence of IL-2. FIG. 2G shows relative % Tyro3⁺ NK cells sorted by CD56^{bright} or CD56^{dim} cells, in the presence or absence of IL-2. One-way ANOVA was used for FIGS. 2A-2D and Pearson correlation coefficient was used for FIG. 2E). P values were adjusted by the Holm-Sidak method. *P<0.05, ***P<0.001; ns, not significant. Data presented as mean±SEM.

FIGS. 3A-3C depict that Tyro3⁻ EGFP fusion protein can be transferred from tumor cells to human NK cells via trogocytosis. FIG. 3A shows representative flow cytometry plots of Axl, Mertk, and Tyro3 expression on parental K562 cells and antigen-presenting K562 (APC-K562) cells. FIG. 3B shows Tyro3 expression on CD56⁺ NK cells co-cultured with K562 or APC K562 at an E/T ratio of 1:1 for 1 hr. Each experiment was performed with 4 different donors. FIG. 3C shows the construct design of the Tyro3⁻ EGFP fusion protein. G4S linker, Gly-Gly-Gly-Gly-Ser (SEQ ID NO:27). FIG. 3D shows representative flow cytometry plots showing co-culture of NK cells with APC K562 cells that overexpress Tyro3 with C-terminal fusion EGFP (APC K562^{Tyro3-EGFP}). Flow cytometry was conducted after 1 hr (1 H), 2 hrs (2 H), or 4 hrs (4 H) of co-culturing. Each experiment was performed with 3 different donors. FIG. 3E shows RT-PCR analysis of Tyro3 expression in NK cells co-cultured with APC K562^{Tyro3-EGFP}). K562 cells were used as positive control for endogenous Tyro3 transcript and served as the negative control for transgenic Tyro3 transcript. Tyro3⁻ and Tyro3⁺ NK cells both lacked the expression of endogenous and transgenic Tyro3 transcripts over the entire 4 hr period of co-culture. Glyceraldehyde 3-phosphate dehydrogenase (GAPDH) was used as a control for quality of cDNA synthesis. Each experiment was performed with 3 different donors.

FIGS. 4A-4D depict functional assessment of unstimulated, Tyro3⁻ and Tyro3⁺ NK cells after NK cells were co-cultured with K562 cells. FIG. 4A depicts flow cytometry plots and summary data from 7 different donors showing expression of CD107a in unstimulated, Tyro3⁻ and Tyro3⁺ NK cells (from left to right in the graph) after NK cells were co-cultured with K562 cells for 24 hrs at an E/T ratio of 10:1. FIG. 4B depicts flow cytometry plots and summary data from 7 different donors showing expression of IFN-γ in unstimulated, Tyro3⁻ and Tyro3⁺ NK cells (from left to right in the graph) after NK cells were co-cultured with K562 cells for 24 hrs at an E/T ratio of 10:1. FIG. 4C depicts a the expression of IFN-γ assessed by qRT-PCR in FACS-sorted unstimulated, Tyro3⁻ and Tyro3⁺ NK cells (from left to right in the graph) after NK cells were co-cultured with K562 cells for 24 hrs at an E/T ratio of 10:1 ratio. Summary data are representative from 5 different donors. FIG. 4D depicts the protein levels of GZMB and perforin assessed by immunoblotting in FACS-sorted Tyro3⁻ and Tyro3⁺ NK cells (from left to right in the graph) after IL-2-stimulated NK cells were co-cultured with K562 cells for 4 hr at an E/T ratio of 1:1 with GolgiPlug™. Summary data are for 3 different donors. FIG. 4E depicts % specific lysis of Tyro3⁻ and Tyro3⁺ NK cells which were FACS-sorted after NK cells were co-cultured with K562 cells for 24 hr at an E/T ratio of 10:1, then co-cultured with ⁵¹Cr labeled-K562 or −721.221 cells for 4 hrs, followed by quantification of specific K562 or 721.221 cell lysis. Data are summarized from 4 different donors. For FIGS. 4F-4I, primary human NK cells were incubated with or without K562 cells for 24 hr at an E/T ratio of 10:1. FIG. 4F depicts summary data of activating receptors (CD25, CD62L, CD69, CD94, TRAIL and NKp80) on unstimulated, Tyro3⁻, and Tyro3⁺ NK cells (from left to right for each group shown in the graph) from 6 different donors. FIG. 4G depicts mean fluorescence intensity (MFI) summary data of KLRG1 on unstimulated, Tyro3⁻, and Tyro3⁺ NK cells (from left to right in the graph) from 6 different donors. FIG. 4H depicts MFI summary data of exhaustion markers (Tim-3 and TIGIT) on unstimulated, Tyro3⁻, and Tyro3⁺ NK cells (from left to right in each graph) NK cells from at least 5 different donors. FIG. 4I depicts % in NK subsets summary data of PD-L1 on unstimulated, Tyro3⁻, and Tyro3⁺ NK cells (from left to right in the graph) from 6 different donors. One-way ANOVA was used for FIGS. 4A-4C and 4G-4I, paired t test for FIG. 4D, multiple t test for FIG. 4E, and two-way ANOVA for FIG. 4F. P values were adjusted by the Holm-Sidak's method. *, P<0.05; **, P<0.01; ***, P<0.001. Data are presented as mean±SD for FIGS. 4A, 4B, 4D and 4F-4I and as mean±SEM for FIGS. 4C and 4E.

FIGS. 5A-5D depict ex vivo NK cell expansion after acquisition of Tyro3 from APC K562 cells expressing Tyro3. FIG. 5A depicts folds change for Tyro3⁻, and Tyro3⁺ NK cells (from left to right in the graph) when Tyro3⁻ and Tyro3⁺ NK cells were FACS-sorted and incubated with inactivated APC K562 cells in the presence of IL-2 (50 IU/ml) for 7 days, followed by enumerating live NK on day 7 (n=9). FIG. 5B depicts folds change for unstimulated, Tyro3⁻, and Tyro3⁺ NK cells (from left to right in the graph) when Tyro3⁻ and Tyro3⁺ NK cells were FACS-sorted and cultured in the presence of IL-2 (150 IU/ml) for 7 days, followed by enumerating live NK on day 7 (n=4). FIG. 5C depicts folds change for NK cells when primary human NK cells were incubated with inactivated APC K562 cells (left column in graph) or APC K562^{Tyro3} (right column in graph) in the presence of IL-2 (50 IU/ml) for 7 days, followed by enumerating live NK on day 7 (n=9). FIG. 5D depicts folds change for NK cells when primary human NK cells were incubated with APC K562 cells (left column in graph) or APC K562^{Tyro3} (right column in graph) for 1 hr. Then NK cells were separated from tumor cells, followed by co-culture with IL-2 (50 IU/ml) for 7 days, followed by enumerating live NK cells on day 7 (n=3). FIG. 5E depicts p-STAT5^{Ser727}, p-AKT or p-ERK expression in NK cells when primary human NK cells were incubated with APC K562 cells (left column in graph) or APC K562^{Tyro3} (right column in graph) for 1 hr. Then NK cells were separated from tumor cells, followed by immunoblotting assay. Summary data are presented after being normalized to β-actin expression. Each experiment was repeated with at least 3 different donors. FIG. 5F depicts Tyro3 expression after IL-2-stimulated primary human NK cells were co-cultured with APC K562^{ED-Tyro3} cells for 1 hr at an E/T ratio of 1:1 (n=3). FIG. 5G depicts folds change in NK cells when primary human NK cells were incubated with APC K562 cells, APC K562^{Tyro3} or APC K562^{ED-Tyro3} (from left to right in graph) in the presence of IL-2 (50 IU/ml) for 7 days, followed by enumerating live NK cells on day 7 (n=5). FIG. 5H shows % BrdU positive NK cells after incubation with APC K562 cells, APC K562^{Tyro3} or APC K562^{ED-Tyro3} (from left to right in graph) as described for FIG. 5G from 3 different donors. FIG. 5I depicts folds change for NK cells when primary human NK cells were incubated with APC K562 cells, APC K562^{Tyro3} or APC K562^{Tyro3_K550A} (from left to right in graph) in the presence of IL-2 (50 IU/ml) for 7 days, followed by enumerating live NK on day 7 (n=6). FIG. 5J depicts Tyro3 expression after IL-2-stimulated primary human NK cells co-cultured with APC K562^{Tyro3_K550A} cells at an E/T ratio of 1:1 (n=3). Paired t test was used for FIGS. 5A, 5C and 5E, two-way ANOVA for FIGS. 5B and 5D, and one-way ANOVA for FIGS. 5G-5I. P values were adjusted by the Turkey's or Holm-Sidak's method. *, P<0.05; **, P<0.01; ***, P<0.001; ****, P<0.0001. Data are presented as mean±SD.

FIGS. 6A-6D depict NK cell in vitro and in vivo effector functions. For FIGS. 4A-4C, primary human NK cells were incubated with inactivated APC K562 cells or APC K562^{Tyro3} cells in the presence of IL-2 (50 IU/ml) for 7 days. FIG. 6A depicts % of NK cells summary data from 3 different donors showing the expression of surface markers on NK cells expanded with APC K562 cells or APC K562^{Tyro3} cells (from left to right for each group in the graph). FIG. 6B depicts representative flow cytometry plots and summary data from 3 different donors showing the expression of CD107a on NK cells expanded with APC K562 cells or APC K562^{Tyro3} cells (from left to right in the graph) after co-culture with parental K562 cells for 4 hr at an E/T ratio of 4:1. FIG. 6C depicts NK cells expanded with APC K562 cells or APC K562^{Tyro3} co-cultured with ⁵¹Cr-labeled K562 cell line for 4 hrs, followed by quantification of specific K562 cell lysis. Data are summarized from 3 different donors. FIG. 6D depicts bioluminescence imaging on days 9 and 14 in mice. Primary human NK cells were incubated with APC K562^{Tyro3} or APC K562 cells in the presence of IL-2 (50 IU/ml) for 4 days. Then cells were transduced with a retrovirus vector expressing soluble IL-15 (sIL15) for 48 h. On day 6, transduced NK cells were stimulated with the second batch of APC K562^{Tyro3} or APC K562 cells in the presence of IL-2 (50 IU/ml) for another 7 days prior to harvest and storage for following usage. On day 0, mice were injected with 1×10⁶ K562_Luc cells and then treated with 10×10⁶ sIL15 NK cells expanded with APC K562 (APC K562_sIL15 NK) or APC K562^{Tyro3} (APC K562^{Tyro3}_sIL15 NK). On day 1, mice were treated with the 2nd dose of these NK cells. N=5 in each group. Two-way ANOVA was used for A. **, P<0.01; ***, P<0.001. Data are presented as mean±SD.

FIG. 7A depicts representative flow cytometry plots showing the percentage of Tyro3⁺ NK cells under different conditions of cytokine stimulation (IL-2 150 IU/ml and all the other cytokines 10 ng/ml) from 4 different donors. NC: No cytokines. FIG. 7B depicts Tyro3, Axl, and Mertk expression after primary human NK cells were co-cultured with K562 at an E/T ratio of 1:1 for 1 hr with/without IL-2-stimulation. Summary data from 3 different donors. FIG. 7C depicts the percentage of Tyro3⁺ expression. EnrNK or FACS-purified NK cells were co-cultured with K562 cells at an E/T ratio of 10:1 for 24 hr, then Tyro3 expression on NK cells was determined by flow cytometry. Data are showing the summarized data from 4 different donors. FIG. 7D shows normalized histograms for expression of Tyro, Axl, and Mer expression on different tumor cell lines (Jurkat, U937, Molm-13, K562, and ISO as shown in the graph from top to bottom). Data are representative of 3 independent experiments. FIG. 7E depicts flow cytometry plots of Tyro3 expression on CD56⁺ NK cells. Primary human NK cells were pre-treated with or without IL-2 (150 IU/ml) overnight, then co-cultured with different leukemia cell lines K562, U937, Molm-13, and Jurkat at an E/T ratio of 1:1 for 1 hr, followed by evaluation of Tyro3 expression on NK cells by flow cytometry. Representative flow cytometry plots from 3 independent experiments are shown. One-way ANOVA for FIG. 7B and paired t test for FIG. 7C. P values were adjusted by the Turkey's method. *, P<0.05; **, P<0.01; ***, P<0.001. Data are presented as mean±SD.

FIG. 8A depicts representative flow cytometry data showing Tyro3 expression on WT and K562^{Tyro3-KO} cells. FIG. 8B depicts representative flow cytometry plots showing Tyro3 expression after co-culture of IL-2-stimulated NK cells with parental Jurkat or Jurkat^{Tyro3-OE} cells at an E/T ratio of 1:1 for 1 hr. FIG. 8C depicts representative flow cytometry plots for the kinetics of Tyro3 acquisition in CD56^{bright} or CD56^{dim} NK cells (after sorting separation). NK cells pre-treated with or without IL-2 (150 IU/ml) were co-cultured with K562 for 1 hr. Representative flow cytometry plots for the kinetics of Tyro3 acquisition in CD56^{bright} or CD56^{dim} NK cells (after sorting separation). Each experiment was performed with 4 different donors.

FIG. 9A depicts expression of granzyme B (GZMB) and perforin by qRT-PCR in FACS-sorted unstimulated, Tyro3⁻ and Tyro3⁺ NK cells (from left to right in each graph) co-cultured with K562 cells for 24 hr at an E/T ratio of 10:1. Summary data of 5 different donors. FIGS. 9B-9C depict representative flow cytometry plots and summary data of 4 different donors showing the expression of CD107a (FIG. 9B) and IFN-γ (FIG. 9C) in unstimulated NK cells, Tyro3⁻ and Tyro3⁺ NK cells (from left to right in each graph) co-cultured with K562 cells for 4 hr at an E/T ratio of 4:1. FIG. 9D depicts summary data of activating receptors (CD16, DNAM-1, NKG2D and NKp30) on unstimulated cells, Tyro3⁻ and Tyro3⁺ NK cells (from left to right in each graph) after NK cells were co-cultured with K562 cells for 24 hr at an E/T ratio of 10:1 from at least 5 different donors. FIG. 9E depicts summary data of NKG2A from 5 different donors on unstimulated NK cells, Tyro3⁻ NK cells and Tyro3⁺ NK cells (from left to right in each graph). One-way ANOVA was used for FIGS. 9A-9C and 9E, and two-way ANOVA for FIG. 9D. P values were adjusted by the Turkey's or Holm-Sidak's method. **P<0.01, ***P<0.001, ****P<0.0001. Data are presented as mean±SD.

FIG. 10A depicts Tyro3 expression after unstimulated primary human NK cells were co-cultured with APC K562 cells which overexpressed Tyro3 without/with inactivation for 1 hr at an E/T ratio of 1:1. Data are summarized from 3 different donors. Primary human NK cells were incubated with inactivated APC K562, APC K562^{Tyro3} or APC K562^{ED-Tyro3} cells in the presence of IL-2 (50 IU/ml) for 7 days. FIG. 10B depicts representative flow cytometry plots and summary data. Expanded NK cells from different feeder cell lines were labeled with carboxyfluorescein diacetate succinimidyl ester (CSFE) dye and then cultured for 24 hr. The groups are APC K562, APC K562^{Tyro3}, or APC K562^{ED-Tyro3} (from left to right). FIG. 10C depicts Annexin V vs Sytox Blue expression in NK cells and summary data about the percentages of apoptotic cells and live cells after NK cells had a 7 day-expansion with different feeder cells. Each experiment was performed with 5 different donors. The groups are APC K562, APC K562^{Tyro3}, or APC K562^{ED-Tyro3} (from left to right for each grouping). FIG. 10D depicts percentage of relative cell numbers of expanded NK cells at 0 hr, 24 hr, 48 hr, and 72 hr (0 H, 24 H, 24 H, and 72 H) in NK cells co-cultured with the APC K562, APC K562^{Tyro3}, or APC K562^{ED-Tyro3} (APC K562, APC K562^{Tyro3}, or APC K562^{ED-Tyro3} cells indicated from left to right in each grouping). Cell counting was performed daily after expanded NK cells were cultured in the absence of IL-2. Each experiment was performed with 3 different donors. FIG. 10E shows expansion fold changes in NK cells. Primary human NK cells were transduced with GFP-EV or GFP-Tyro3, followed by culture in the presence or absence of IL-2. FACS-sorted GFP⁺ or Tyro3⁺ transduced cells were incubated without or with IL-2 (150 IU/ml) for 96 hr. Cell counting was performed daily. Data shown are the summary of 4 different donors. One-way ANOVA was used for FIGS. 10A and 10B, and two-way ANOVA for FIGS. 10C-10E. P values were adjusted by the Holm-Sidak's method. *, P<0.05; **, P<0.01; ***, P<0.001. Data are presented as mean±SD.

FIG. 11A depicts representative flow cytometry plots showing Tyro3 expression in human NK cells from different organs of mice. Each flow plot is representative of data from 4 mice with similar results. FIG. 11B depicts RT-PCR analysis of Tyro3 expression in human NK cells from different organs or tissues of mice. The K562 cell line was used as a positive control for the endogenous Tyro3 at the mRNA level. All human NK cell samples lacked the expression of endogenous Tyro3 transcript. GAPDH was used as a control for the quality of cDNA synthesis. Each experiment was performed twice. BM, bone marrow; LV, liver; SP, spleen; PB, peripheral blood.

### DETAILED DESCRIPTION

Unless otherwise defined herein, scientific and technical terms used in this application shall have the meanings that are commonly understood by those of ordinary skill in the art. Generally, nomenclature used in connection with, and techniques of, cell and tissue culture, molecular biology, cell and cancer biology, virology, immunology, microbiology, genetics and protein and nucleic acid chemistry, described herein, are those well-known and commonly used in the art. In case of conflict, the present specification, including definitions, will control.

The practice of the present application will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al, 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al, eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, NY (2001); Ausubel et al., Current Protocols in Molecular Biology, John Wiley & Sons, NY (2002); Harlow and Lane Using Antibodies: A Laboratory Manual, Cold Spring Harbor Laboratory Press, Cold Spring Harbor, NY (1998); Coligan et al., Short Protocols in Protein Science, John Wiley & Sons, NY (2003); Short Protocols in Molecular Biology (Wiley and Sons, 1999).

Throughout this specification and embodiments, the word “comprise,” or variations such as “comprises” or “comprising,” will be understood to imply the inclusion of a stated integer or group of integers but not the exclusion of any other integer or group of integers. It is understood that wherever embodiments are described herein with the language “comprising,” otherwise analogous embodiments described in terms of “consisting of’ and/or “consisting essentially of’ are also provided.

The term “including” is used to mean “including but not limited to”. “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a”, “an” and “the” are used herein to refer to one or to more than one (i.e., to at least one) of the grammatical object of the article. By way of example, “an element” means one element or more than one element. Reference to “about” a value or parameter herein includes (and describes) embodiments that are directed to that value or parameter per se. For example, description referring to “about X” includes description of “X.” Numeric ranges are inclusive of the numbers defining the range. As used herein, the term “about” permits a variation of ±10% within the range of the significant digit.

Notwithstanding that the disclosed numerical ranges and parameters are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.3, and ending with a maximum value of 10 or less, e.g., 5.7 to 10.

Where aspects or embodiments are described in terms of a Markush group or other grouping of alternatives, the present application encompasses not only the entire group listed as a whole, but each member of the group individually and all possible subgroups of the main group, and also the main group absent one or more of the group members. The present application also envisages the explicit exclusion of one or more of any of the group members in the Markush group or other grouping of alternatives.

### Definitions

As described herein, the term “modified to exogenously express” refers to forcing the expression of a gene of interest in a cell. In the context of this disclosure, Tyro3 is exogenously expressed in feeder cells (such as K562 cells). “Exogenous expression” refers to the forced surface expression or “overexpression” of the protein of interest (Tyro3; SEQ ID NO:2) or a protein that is at least 95% identical to SEQ ID NO:2 on the surface of feeder cells. In order to induce exogenous expression of Tyro3, the coding sequence for Tyro3 (SEQ ID NO:1) or a nucleic acid that is at least 95% identical to the SEQ ID NO:1 may be cloned into an expression vector and delivered to the K562 cells by plasmid transfections or lentiviral particle transduction. The Tyro3 delivered to the K562 may be under the control of a “strong promoter”, which is a promoter that leads to a high level of transcription of mRNA. In some instances, the strong promoter may be a “constitutive promoter”, which is an unregulated promoter that is active under all circumstances, and allows for continual transcription of its associated gene. In some instances, the strong promoter is a retroviral promoter, or any other promoter known in the art.

As described herein, the term “expanding a population of cells” refers to the process of culturing cells in vitro or ex vivo by conventional cell culture methods known in the art. In the context of this disclosure, culturing cells or “cultivation” includes the step of “co-culturing” human NK cells with Tyro3⁺ feeder cells (Tyro3⁺ K562 cells), by the methods of the disclosure. Culturing provides the chemical and physical conditions (e.g., temperature, gas, pressure, etc.) which are required for NK cell and feeder cell maintenance, as well as growth factors. Culturing the NK cells includes providing the NK cells with conditions for expansion or proliferation. Examples of chemical conditions which may support NK cell expansion include but are not limited to buffers, serum, nutrients, vitamins, antibiotics, cytokines and other growth factors which are regularly provided in (or may be given manually to) the cell culture medium suited for NK cell expansion.

In one embodiment, the NK cell culture medium includes TexMACS Research Medium (Miltenyi Biotec GmbH) supplemented with 0-20% human serum type AB (Life Technologies) and 0-2000 IU/mL of interleukin-2 (IL-2) (Proleukin S, Novartis). In another embodiment the NK cell culture medium includes Stem Cell Growth Medium SCGM (Cell Genix) supplemented with 0-20% human serum type AB (Life Technologies) and 0-2000 IU/mL of IL-2 (Proleukin S, Novartis). Other media suitable for use in expanding NK cells are well known in the art.

Cell culture media or liquids providing the chemical conditions which are required for NK cell and feeder cell maintenance. Examples of chemical conditions which may support NK cell and feeder cell maintenance, as well as NK cell expansion include but are not limited to solutions, buffers, serum, serum components, nutrients, vitamins, cytokines and other growth factors which are regularly provided in (or may be given manually to) the cell culture medium. Media suitable for use to cultivate NK cells as known in the art include TexMACS (Miltenyi), CellGro SCGM (CellGenix), X-Vivo 10, X-Vivo 15, BINKIT NK Cell Initial Medium (Cosmo Bio USA), AIM-V (Invitrogen), DMEM/F12, NK Cell Culture Medium (Upcyte Technologies).

As used herein, the term “expansion”, “proliferation”, “multiplication” or cognates thereof refer to the increase of cell numbers during cell culture. During culture, the cells undergo a series of cell divisions and thus expand in numbers. Expansion, as used herein relates to increased numbers of Tyro3⁺ NK cells occurring during the cell culture process disclosed in the methods of the disclosure. In one embodiment, the term “expanded NK cells” refers to a group of activated NK cells, which cells (a) acquire Tyro3 on their surfaces via trogocytosis from Tyro3⁺ feeder cells (e.g., Tyro3⁺ K562 cells cells) during cell culture; (b) are sorted via techniques known in the art (e.g., Fluorescence Activated Cell Sorting or FACS) to yield a pure population of Tyro3⁺ CD56⁺ NK cells, and (c) the Tyro3⁺ CD56⁺ NK cells are subsequently expanded in cell culture in the presence of IL-2. The expanded NK cell population may subsequently lose Tyro3 expression from the cell surface. However, the expanded NK cells may still retain their activation status and cytotoxic functions, i.e., the expanded NK cells produced by the methods of the disclosure may have enhanced cytotoxicity and higher expression of activated surface markers when compared with resting NK cells, NK cells, and/or Tyro3⁻ NK cells. The NK cells of the disclosure may be expanded in a specific cGMP grade environment and cGMP grade medium.

As used herein, the term “primary NK cells” refer to NK cells that may be obtained from any conventional source such as from peripheral blood, bone marrow, cord blood, induced pluripotent stem cells (iPSCs), cell lines, cytokine stimulated peripheral blood, etc using techniques known in the art. See e.g., Fang F, et al. Cancer Biol Med. 2019; 16:647-54. doi: 10.20892/j.issn.2095-3941.2019.0187. The primary NK cells are immune cells with typical NK cell markers such as CD56 isolated from healthy donors or patients. In some embodiments, a primary NK cell may be a cell that has previously been in contact with a Tyro3⁺ feeder cell but did not acquire Tyro3 via trogocytosis, or lost expression of Tyro3⁺ after being purified or FACS sorted.

As described herein, the term “trogocytosis” (also known as shaving) refers to a process of fast, cell-to-cell contact-dependent uptake of membrane patches and associated molecules between two different types of live cells, e.g., from a feeder cell to an NK cell in the methods of this disclosure. As a result, the membrane-bound proteins and membrane components are transferred from the donor cells (feeder cells) to the recipient cells (NK cells). When an NK cell interacts with a feeder cell, such as a K562 cell, an immune synapse forms strong enough to allow for the exchange of small membrane molecules from the feeder cell to the NK cell. The protein Tyro3 is transferred via trogocytosis from a Tyro3⁺ feeder cell onto the surface of a human NK cell in the methods of this disclosure.

Without being bound by theory, it is believed that trogocytosis of Tyro3 from feeder cells to NK cells in the methods of this disclosure surprisingly enhances the ability of NK cells to proliferate, and also enhances the activation and cytotoxic functions of the expanded NK cells.

The phenotype and biological function consequence of trogocytosis in recipient cells can be different (Campana S, et al. Immunol Lett. 2015; 168(2):349-54). The IL-2-activated NK cell line NKL became suppressive NK cells after acquiring HLA-G1 from an HLA-G1-transfected melanoma cell line (Caumartin J, et al. EMBO J. 2007; 26(5):142). A reduction in NK cell cytotoxicity was observed after the intracellular transfer of NKG2D from NK cells to target cells (Roda-Navarro P, et al. Proc Natl Acad Sci USA. 2006; 103(30):11258-63). By contrast, as shown in the Examples of this disclosure, upon encountering and being activated by NK-susceptible tumor cells (K562), Tyro3⁺ NK cells were found to have significantly enhanced cytotoxicity, IFN-γ production and expression of activated surface markers compared to Tyro3⁻ NK cells. Further, the expression of PD-L1, which has been shown to be upregulated on NK cells after encountering with K562 cells (Dong W, et al. 2019; 9(10):1422-37), increases in the Tyro3⁺ NK cell subset compared to the Tyro3⁻ NK cell subset.

As used herein, the term “Tyro3+ NK cells” refers to NK cells whose outer surfaces comprise Tyro3 protein, or peptide derivatives thereof. The majority of Tyro3 protein or peptide derivatives thereof on the NK cell surfaces (>98%) may be obtained from the K562 cells or any encountering cells via trogocytosis rather than being encoded and expressed by mRNA within the cell. Thus, very little, if any (i.e., less than 5%), of the Tyro3 present on the surface of Tyro3+ NK cells is produced endogenously by the NK cell itself.

As used herein, the term “Tyro3⁺ K562 cells” are engineered K562 cells in which Tyro3 protein expressed on the cell surface is synthesized in the K562 cell by forced expression of Tyro3. The Tyro3 expression may be under the control of a strong promoter.

Thus, in the context of this disclosure, the term “Tyro3⁺” when used for the NK cell refers to NK cells that acquire Tyro3 on their outer cell surfaces, which surface expression is not encoded by endogenous mRNA. By contrast, the term “Tyro3⁺” when used for the modified K562 cell line or other feeder cell line (e.g., Molm-13 or U937) refers to the forced expression of Tyro3 from an expression vector comprising a sequence encoding Tyro3 delivered to the K562 cells or other feeder cells.

The terms “engineered cell” and “genetically modified cell” as used herein can be used interchangeably. The terms mean containing and/or expressing a foreign gene or nucleic acid sequence which in turn modifies the genotype or phenotype of the cell or its progeny. In particular, the terms refer to the fact that cells can be manipulated by recombinant methods well known in the art to express stably or transiently peptides or proteins which are not expressed in these cells in the natural state. Genetic modification of cells may include but is not restricted to transfection, electroporation, nucleofection, transduction using retroviral vectors, lentiviral vectors, non-integrating retro- or lentiviral vectors, transposons (e.g., Sleeping Beauty Transposon System), designer nucleases including zinc finger nucleases, TALENs or CRISPR/Cas. In one embodiment, the genetically modified NK cell may express a chimeric antigen receptor (CAR). CARs are able to redirect a cytotoxic immune response against all cells that express the antigen they bind to. CARs may be used in the cancer immunotherapy, wherein NK cells, carrying a CAR targeted to a tumor antigen may generate a strong antitumor response against cells expressing the antigen targeted by the CAR (Sadelain et al., Cancer Discovery. 2013. 3(4):388-98). In some embodiments CAR-NK cells may be expanded using methods of the disclosure. “Activated Tyro3⁺ NK cells” refer to NK cells of the disclosure that were expanded in the presence of Tyro3⁺ feeder cells (such as K562 cells) and are thereby activated and acquire a cytotoxic phenotype and have enhanced NK cell function. A number of markers of activation, and cytotoxic function may be upregulated on the surface of the expanded NK cell population produced by the methods of this disclosure. The activating pathway of NK cells also includes a series of different receptors. Activating receptors do not directly signal through their cytoplasmic tail, but instead associate non-covalently with other molecules containing ITAMs (immunoreceptor tyrosine-based activation motifs), that serve as the signal transducing proteins. Thus, according to one embodiment, the ex vivo expanded and activated NK cells have an upregulated expression of at least one activating receptor. Activated Tyro3⁺ NK cells may upregulate cell surface activation markers, e.g., CD25 and/or CD69, and upregulate killer cell lectin-like receptor G1 (KLRG1), which is expressed on the most mature NK cells and is a receptor for NK cell maturation. In some instances, such cells upregulate the expression of IFNγ and/or CD107a, which are functional markers for NK cell degranulation and cytokine production, following NK cell activation. In addition, activated Tyro3⁺ NK cells may upregulate PD-L1 expression which is an indication of enhanced NK cell function. Activated Tyro3⁺ NK cells of this disclosure may have enhanced expression and activity of cell signaling molecules such as p-STAT3, p-P65, p-STAT5, pAKT, p-ERK, etc.

In some embodiments, the mRNA or protein levels of IFNγ and/or CD107a in activated Tyro3⁺ NK cells are greater than those in a control by at least about 2.0× or 3.0× or 4.0× or 5.0× or 6.0× or 7.0× or 8.0× or 9.0× or 10×, or 20× or 50×, or 75× or greater than about 100×. In some embodiments, the mRNA or protein levels of IFNγ in activated Tyro3⁺ NK cells are greater than those in a control by at least about 50×, or 100×, or 250×, or 500×, or 750×, or 1000×, or 2000× or greater than about 2000×. In some embodiments, the mRNA or protein levels of cell surface activation markers, cell signaling molecules, KLRG1, and/or PD-L1 in activated Tyro3⁺ NK cells is greater than that in a control by at least about 2.0× or 3.0× or 4.0× or 5.0× or 6.0× or 7.0× or 8.0× or 9.0× or 10×, or 20× or 50× or greater than about 100×. The fold change in mRNA levels may be measured by any methods known in the art including but not limited to Northern blot or dot blot analysis, reverse transcriptase-PCR (RT-PCR; e.g., quantitative RT-PCR), in situ hybridization (e.g., quantitative in situ hybridization) or nucleic acid array (e.g., oligonucleotide arrays or gene chips) analysis. Details of such methods are in, e.g., Sambrook et al., Molecular Cloning: A Laboratory Manual Second Edition vol. 1, 2 and 3. Cold Spring Harbor Laboratory Press: Cold Spring Harbor, New York, USA, November 1989; Gibson et al. (1999) Genome Res., 6(10):995-1001; and Zhang et al. (2005) Environ. Sci. Technol., 39(8):2777-2785; U.S. Publication No. 2004086915; European Patent No. 0543942; and U.S. Pat. No. 7,101,663; the disclosures of each of which are incorporated herein by reference in their entirety. The fold change in protein levels may be measured by any methods known in the art including, but not limited to, western blot or dot blot analysis, immunohistochemistry (e.g., quantitative immunohistochemistry), immunocytochemistry, enzyme-linked immunosorbent assay (ELISA), enzyme-linked immunosorbent spot (ELISPOT; Coligan, J. E., et al., eds. (1995) Current Protocols in Immunology. Wiley, New York), radioimmunoassay, chemiluminescent immunoassay, electrochemiluminescence immunoassay, latex turbidimetric immunoassay, latex photometric immunoassay, immuno-chromatographic assay, and antibody array analysis (see, e.g., U.S. Publication Nos. 2003/0013208 and 2004/171068, the disclosures of each of which are incorporated herein by reference in their entirety). Further description of many of the methods above and additional methods for detecting protein expression can be found in, e.g., Sambrook et al. In some embodiments, fold change in cell surface protein levels of a marker in activated Tyro3⁺ NK cells compared with a control (e.g., unstimulated NK cells, Tyro3⁻ cells, etc.) may be measured by determining the mean fluorescence intensity (MFI) of the marker of interest by flow cytometry as described in the “Examples”.

The NK cells of this disclosure are expanded, active and have a phenotype cytotoxic against tumor cells, preferably autologous tumor cells.

As used herein, the term “control” refers to resting NK cells, primary NK cells, unstimulated NK cells, and/or Tyro3⁻ NK cells depending on the context.

In the content of the present disclosure, NK cells that have a “cytotoxic phenotype” relates to cells that are cytotoxic, i.e. they induce the death of other cells such as, but not limited to, tumor cells, virus-infected cells or cells that are otherwise damaged or dysfunctional. Cytotoxic cells of the present disclosure are mainly toxic to tumor cells and virus-infected cells. The cytotoxicity of NK cells towards cells can easily be measured, for example, by traditional cell counting before and after exposure to the expanded NK cells of the disclosure. Such methods are well known to a person skilled in the art. Examples of suitable methods are, but not limited to, fluorescent cell counting assay, immunofluorescent cell counting assay, Chromium-51 release assay, cell viability assay, and flow cytometry-based cytotoxicity assay.

As used herein, the term “cell surface density” refers to the surface expression of a particular protein (e.g., Tyro3) on the outer membrane surface of a cell (e.g., an NK cell). The cell surface density may be measured directly or indirectly by methods known in the art, including but not limited to flow cytometry (Robins R. A. (1998) In: Pound J. D. (eds) Immunochemical Protocols. Methods in Molecular Biology™, vol 80. Humana Press.; Donnenberg, V. S., et al. Cytometry, 93: 803-810. doi:10.1002/cyto.a.23525), super-resolution optical fluctuation imaging (Lukes T et al., Nat Commun. 2017; 8: 1731), liquid scintillation counting, fluorescence subtraction, fluorescence correlation spectroscopy (Chen Y et al., Chemistry. 2009; 15(21): 5327-5336), etc. When using flow cytometry, cell surface density may be determined by measuring mean fluorescence intensity as described by methods known in the art. See e.g., Robins R A. Methods Mol Biol. 1998; 80:319-36 and Moskalensky A E, et al. J Immunol Methods. 2015 March; 418:66-74. In some instances, geometric mean fluorescence intensity is obtained and converted into antigen density for each protein or marker and plotted on a log scale. Cell surface density of a protein or marker on the cell surface can also be determined with lipid-encapsulated fluorescent nanodiamonds of 35 nm in diameter as biolabels as described in Hsieh F J, et al. Micromachines (Basel). 2019; 10(5):304. Using the techniques described above, the cell surface density range for Tyro3 on the membrane surface can be obtained. Tyro3 cell surface density may range from about 10, about 100, about 1000, about 10⁴, about 10⁵, about 10⁶, or about 10⁷ Tyro3 antigens/cell.

As used herein, a “composition” refers to a preparation of the expanded NK cells of the disclosure. The composition may have a physiologically acceptable carrier, diluent, excipient, and/or other components such as IL-2 or other cytokines or cell populations. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. A composition of the disclosure may comprise a) a population of NK cells, wherein said NK cells are expanded to therapeutically effective amounts; and b) one or more pharmaceutically or physiologically acceptable carriers, diluents or excipients. Administration of the NK cells in the compositions of the disclosure can be autologous or heterologous. For example, NK cells can be obtained from one subject, and administered to the same subject or a different, compatible subject. Compositions of the disclosure can be formulated for administration via localized injection, including catheter administration, systemic injection, localized injection, intravenous injection, or parenteral administration. When administering a therapeutic composition of the present invention (e.g., a pharmaceutical composition containing a genetically modified immunoresponsive cell), it will generally be formulated in a unit dosage injectable form (solution, suspension, emulsion).

Methods of administering compositions comprising cells by adoptive immunotherapy are known in the art and include procedures such as those exemplified in U.S. Pat. Nos. 4,844,893 and 4,690,915 and International patent Application No. PCT/US2014/018667.

Compositions of the present disclosure may be administered in a manner appropriate to the condition to be treated. The quantity and frequency of administration will be determined by such factors as the condition of the subject in need thereof, and the type and severity of the subject's condition, although appropriate dosages may be determined by clinical trials. The expanded NK cells achieved with the methods of this disclosure may be used in subsequent therapeutic or non-therapeutic applications.

### NK Cells

Natural killer (NK) cells comprise 5% to 20% of human peripheral blood lymphocytes and are derived from CD34⁺ hematopoietic progenitor cells. NK cells have the morphology of large granular lymphocytes, and are phenotypically defined by the expression of CD56 and the lack of CD3 and T-cell receptor molecules. NK cells function predominantly in direct cytotoxicity and antibody-dependent cellular cytotoxicity (ADCC). The function of NK cells is regulated by the balance between activation and inhibitory signals. Upon encountering normal cells, NK cells recognize their major histocompatibility complex (MHC) class I molecules and induce inhibitory signals to override activating signals (Gasser S, and Raulet D H. Immunol Rev. 2006; 214:130-42). By contrast, NK cells exert strong cytotoxic effects when they encounter cells lacking self-MHC class I molecules via multiple mechanisms including releasing cytotoxic granules such as perforins and granzymes (Liao N S, et al. Science. 1991; 253(5016):199-202).

NK cells differ from natural killer T cells (NKTs) phenotypically, by origin and by respective effector functions; often, NKT cell activity promotes NK cell activity by secreting IFNy. In contrast to NKT cells, NK cells do not express T-cell antigen receptors (TCR) or pan T marker CD3 or surface immunoglobulins (Ig) B cell receptors, but they usually express the surface markers CD 16 (FcyRIII) and CD56 in humans.

### Expansion of NK Cells

Natural killer cell-based immunotherapy used to treat cancer requires the adoptive transfer of a large number of activated NK cells. Obtaining sufficient numbers of activated NK cells is important for an effective NK cell-based immunotherapy (Kweon S et al., Front. Immunol., 24) April 2019).

NK cells can be expanded in vitro by cultivating with combinations of cytokines, by supplementing cell culture media with small molecules (e.g. Nicotinamide) and by combinations of cytokines, antibodies and feeder cells. However, cytokine-based NK cell cultures often result in only a minor increase in cell numbers that are not sufficient to manufacture NK cell products for multiple patients or from small NK cell subpopulations. NK cells may stop growing in these protocols after 3 weeks, and a prolonged culture does not always result in higher NK cells numbers. Feeder cell-based NK cell expansion protocols generate higher NK cell numbers. However, there is a need for effective protocols that would predictably produce expanded numbers of NK cells that would be of therapeutic value. The present disclosure provides a novel trogocytosis-based in vitro expansion method for NK cells, which cells acquire Tyro3 from the surface of feeder cells. The methods of this disclosure may be used to generate NK cell numbers which are sufficient for clinical application of the expanded NK cells in patients.

The methods of this disclosure can take place in any container compatible with cell culture and expansion, e.g., flask, tube, beaker, dish, multiwell plate (e.g., G-REX®), bag or the like. In a specific embodiment, the co-culturing of NK cells with feeder cells takes place in a bag, e.g., a flexible, gas-permeable fluorocarbon culture bag (for example, from American Fluoroseal). In a specific embodiment, the container in which the NK cells are cultured is suitable for shipping, e.g., to a site such as a hospital or military zone wherein the expanded NK cells are further expanded.

### Cell Lines

Novel cell lines of this disclosure are feeder cells or donor cells that are added to a culture of NK cells to support NK cell survival and/or growth. Feeder cells of the disclosure aid in the expansion of NK cells ex vivo or in vitro. Feeder cells provide an intact and functional extracellular matrix and matrix-associated factors and secrete known and unknown cytokines into the conditioned medium. Feeder cells are usually growth arrested to prevent their proliferation in the culture, but their survival is maintained. Growth arrest can be achieved by irradiation with an effective dose or treatment with an effective dose of chemicals such as Mitomycin C. In the context of this disclosure, feeder cells useful in the methods disclosed herein include but are not limited to cancer cell lines (e.g., chronic myelogenous leukemia (CML) cells such as K562 etc), fibroblasts, stem cells (e.g., stem cells), blood cells (e.g., allogeneic or autologous irradiated or non-irradiated peripheral blood mononuclear cells (PBMC) depleted of NK cells), genetically engineered cancer cell lines, lymphocytes immortalized by natural infection with Epstein-Barr Virus (EBV), etc. Preferably, the feeder cells are K562 cells. The K562 cells may be genetically engineered to express certain ligands, for instance OX40 ligand (Kweon S et al., Front. Immunol., 24 Apr. 2019). K562 cells expressing membrane bound IL-21 and 41-BB Ligand (APC K562) are preferably used in the methods of this disclosure. An APC K562 cell line can be generated from commercially available K562 cells by methods known in the art. Other cell lines useful in the disclosed methods are K562-s (ATCC® CRL-3343™), K562 (ATCC® CCL-243™), K562-s (ATCC® CRL-3344™) and cell lines described in U.S. Pat. Nos. 7,435,596, 8,026,097, 9,623,082, and 10,463,715, the disclosures of each of which are incorporated herein by reference in their entirety. Apart from K562 cells, other cell lines, such as an MHC class I negative or low cell line (e.g., the human B-lymphoblastoid cell-line 721.221, the Acute Myeloid Leukemia-3 (AML3) cell line, or a melanoma cell like lacking MHC Class I) can also be modified to express membrane bound IL-21 and 41-BB Ligand for use in the methods of this disclosure. Such cell lines are well known in the art. See e.g., Dong W et al., Cancer Discov. 2019 October; 9(10):1422-1437 and Porgador A, et al. Proc Natl Acad Sci USA. 1997 Nov. 25; 94(24):13140-5.

Any feeder cell of the present disclosure may be engineered by methods known in the art to exogenously express Tyro3. The Tyro3 synthesized in a feeder cell by forced expression is then displayed on the feeder cell surface. In certain embodiments, feeder cells are optionally inactivated by irradiation (e.g., γ-irradiation) or treatment with an anti-mitotic agent such as mitomycin C, to prevent them from outgrowing the cells they are supporting, but permit synthesis of important factors that support the NK cells. For example, feeder cells can be irradiated at a dose to inhibit their proliferation but permit synthesis of important factors that support NK cells.

In addition to Tyro3, membrane bound IL21, and 41-BBL, any feeder cell of the present disclosure may be engineered to express one or more of CD64 (FcγRI), CD86 (B7-2), and truncated CD19 (tCD19). In some embodiments, the feeder cells express membrane bound IL15, soluble IL15 or an IL15 sushi receptor complex. Such molecules are known in the art. See e.g., Ye et al. Journal of Translational Medicine 2011, 9:131; and Rushworth D. et al., J Immunother. 2014 May; 37(4): 204-213; Suhoski M M, et al. Mol Ther. 2007 May; 15(5):981-8; Wei X, et al. J Immunol. 2001 Jul. 1; 167(1):277-82; and Hu, Q., et al. Sci Rep 8, 7675 (2018). The sequences for these genes and proteins are as follows: CD64 protein sequence: UniProtKB ID P12314 (SEQ ID NO:28). CD64 nucleic acid sequence: NM_000566.3 (SEQ ID NO:29). CD86 (B7-2) protein sequence: UniProtKB P42081 (SEQ ID NO:30). CD86 (B7-2) nucleic acid sequence: NM_175862.4 (SEQ ID NO:31). tCD19 protein sequence: amino acid 1-313 of NP_001171569.1 (SEQ ID NO:32. CD19 nucleic acid sequence: the nucleic acid sequence of NM_001178098.2 (SEQ ID NO:33) representing amino acids 1-313 of SEQ ID NO:32. IL15 protein sequence: UniProtKB P40933 (SEQ ID NO:34). IL15 nucleic acid sequence: NM_000585.4 (SEQ ID NO:35).

### Tyro3

Tyro3 belongs to the TAM (Tyro3, Axl and Mertk) receptor family which is group of receptor tyrosine kinases (RTKs) (Rothlin C V, Annu Rev Immunol. 2015; 33:355-91). In hematopoietic cells, TAM receptors are primarily expressed by cells of the innate immune system, such as macrophages (Zagorska A, et al. Nat Immunol. 2014; 15(10):920-8) and DCs (Carrera Silva E A, et al. Immunity. 2013; 39(1):160-70). Mice NK cells have a high expression of TAM receptors, while human NK cells only yield a faint band of Mer by PCR methods (Paolino M, et al. Nature. 2014; 507(7493):508-12; Park I K, et al. Blood. 2009; 113(11):2470-7). Notably, human NK cells do not express Tyro3 endogenously, or express Tyro3 only in trace amounts. Studies show that Tyro3 is expressed in different types of cancers and play important roles in cancer progression. Therefore, TAM family receptors are considered ideal therapeutic targets (Chen D, et al. Aging (Albany NY). 2020; 12(3):2261-74; Tsai C L, et al. Clin Cancer Res. 2020; 26(5):1185-97; Morimoto M, et al. Cancer Lett. 2020; 470:149-60).

### Methods of Treatment

The methods described herein can be used to produce an expanded population of Tyro3⁺ NK cells compared to methods known in the art. The cells thus produced may be used to treat cancer and suppress the proliferation of tumors due to the cytotoxic activity of NK cells. For instance, the expanded Tyro3⁺ NK cells may be used to treat a wide range of cancers including by not limited to lung cancer, breast cancer, ewing sarcoma, central nervous system neoplasm, skin cancer, head and neck cancer, ovarian cancer, colon cancer, anal cancer, stomach cancer, gastrointestinal cancer, fallopian tube cancer, endometrial cancer, cervical cancer, vaginal cancer, vulvar cancer, esophageal cancer, endocrine cancer, thyroid cancer, parathyroid cancer, adrenal cancer, soft tissue sarcoma, urethral cancer, penile cancer, testicular cancer, brain stem glioma, pituitary cancer, adrenocortical cancer, gallbladder cancer, multiple myeloma, cholangiocarcinoma, fibrosarcoma, lymphoma, liver cancer, kidney cancer, bone cancer, bladder cancer, colorectal cancer, endometrial cancer, renal cell cancer, pancreatic cancer, prostate cancer, thyroid cancer, mesothelioma, neuroblastoma, retinoblastoma, melanoma, rhabdomyosarcoma, leukemia and lymphoma.

The cells of this disclosure may also be used to treat acute or chronic viral infections, such as infections caused by human immunodeficiency virus (HIV), Epstein-Barr virus (EBV), herpes simplex virus (HSV), cytomegalovirus (CMV), varicella-zoster virus (VZV), hepatitis B virus (HBV) or hepatitis C virus (HCV), coronavirus, etc. In one embodiment, the expanded Tyro3⁺ NK cells of this disclosure may be used to treat COVID-19 caused by SARS-CoV-2.

Additionally, Tyro3⁺ NK cells can express an antitumor or antiviral chimeric antigen receptors (CAR-NK). Engineered NK cell therapy, such as Tyro3⁺ CAR-NK cell-based immunotherapy may be used in cancer therapy or anti-viral therapy. For instance, Tyro3⁺ CAR-NK cell line therapy may provide a favorable treatment alternative to relapsed and refractory B cell malignancies (Ochsner J. 2019 Fall; 19(3): 186-187.)

In some embodiments, the NK cell to be expanded using the methods of this disclosure is a CAR-modified NK cell, wherein the CAR is specific for any one target from a wide range of targets, including but not limited to CAR targets for hematological malignancies such as CD19, CD20, CD22, CD30, CD33, CD38, CD70, CD123, Kappa, NKG2D ligands, ROR1; CAR targets for solid tumors such as B7H3 CD44 v6/v7, CD171, CEA, EGFRvIII, EGP2, EGP40, EphA2, ErbB2(HER2), ErbB receptor family, ErbB3/4, HLA-A1/MAGE1, HLA-A2/NY-ESO-1, FR-α, FAP, FAR, GD2, GD3, HMW-MAA, IL11Rα, IL13Rα2, Lewis Y, Mesothelin, Muel, PSCA, HPB, PSMA, TAG72, and VEGFR-2. Such targets are well known in the art. See e.g., Dotti, et al., Immunol Rev. 2014 January; 257(1); and U.S. Pat. Nos. 10,765,701, 10,155,038, 10,577,417, 10,799,536, the disclosures of each of which are incorporated herein by reference in their entirety.

Tyro3⁺ NK cells and Tyro3⁺ CAR-NK cells of the present disclosure may function as immune adjuvants with the ability to boost the efficacy of anticancer therapy when combined with traditional treatments such as chemotherapy (Hu W et al., Front Immunol. 2019; 10: 1205). Further, the NK cells of this disclosure may also be used to produce extracellular vesicles (EVs) that be applied in cancer therapies. EVs are nano-sized vesicles with anti-tumor activity that are naturally secreted by NK cells and provide a cell-free immunotherapy avenue (Hu W et al., Front Immunol. 2019; 10: 1205).

Tyro3⁺ NK cells and Tyro3⁺ CAR-NK cells of the present disclosure may be administered alone or used in combination with other cancer therapies, such as, but not limited to surgery, radiation and cytotoxic drugs. Similarly, the NK cells of the present disclosure may be administered alone or used in combination with other antiviral therapies. The NK cells of this disclosure may be administered prior to, at the same time as, or subsequent to the cancer therapy or anti-viral therapy.

The expanded Tyro3⁺ NK cells and Tyro3⁺ CAR-NK cells of the present disclosure may be administered to a subject in need thereof by adoptive cell transfer (ACT) wherein the NK cells may have originated in the same subject or a different subject. Further, the expanded NK cells of the disclosure can used for adoptive immunotherapy in conditions such as cancer and viral infections. For instance, autologous ex vivo expanded Tyro3⁺ NK cells and Tyro3⁺ CAR-NK cells, can be administered, either prophylactically or therapeutically, to patients undergoing autologous hematopoietic stem cell transplantation for diseases such as multiple myeloma which have in general a poor prognosis with high incidence of progressive disease post transplant. Ex vivo expanded Tyro3⁺ NK cells and Tyro3⁺ CAR-NK cells of donor origin can be used for the treatment of recurrent malignant disease following allogeneic stem cell transplantation. Autologous ex vivo expanded NK cells can be administered, either prophylactically or therapeutically, to patients undergoing autologous stem cell transplantation for cancer. Other examples include using autologous expanded Tyro3⁺ NK cells for ex vivo purging of malignant cells in the harvest, for treatment of patients with hematological malignancies, and as a cellular therapy for solid tumors.

The expanded NK cells of the disclosure can also be administered to a patient in order to prevent recurrence of a malignant disease. In one instance, a sample (e.g., from peripheral blood, bone marrow, or cord) is taken from a patent afflicted with a malignant disease. In one embodiment, the patient has been treated with conventional cancer therapies but the treatment has been unsuccessful or the malignancy has recurred. The method can also be a prophylactic treatment, for example, to prevent recurrence of a malignant disease. Before culturing the primary NK cells with Tyro3⁺ feeder cells, the primary NK cells are purified and separated according to methods well known in the art. The cells are thereafter ex vivo or in vitro expanded in accordance with the methods of this disclosure and in the “Examples”. Subsequently, the expanded cells are administered to the patient. The patient is thereafter carefully followed-up in order to determine if the patient has responded well to the treatment and to determine if the treatment is to be repeated. Samples can, for example, be taken from blood, bone marrow and or urine for follow-up of the treatment at regular time intervals.

In the method of this disclosure, the NK cells are expanded ex vivo for at least about 1 day, 4 days, 8 days, 12 days, 16 days, 20 days, 24 days, 28 days, or greater than about 28 days. Preferably, the NK cells have been expanded ex vivo for about 6-28 days, before administration to the patient. In another embodiment the NK cells have been expanded at least about 50 fold to about 50,000 fold compared to day 0 of expansion, before administration to a patient.

The method of treatment of the disclosure may be performed once or repeated several times. In one embodiment the expanded NK cells of the disclosure are administered to the patient as needed, about 1-10 times, preferably about 1-7 times, more preferably about 1-5 times and most preferably about 1-3 times. The administration route can be any suitable way of administration well known to the skilled person for example, but not limited to; intravenous, intraperitoneal and intratumoral administration. The dosage can be the same in all administrations or for example high in the first administration(s) and then lower in subsequent administrations. The therapies can be a monotherapy or combinational therapies with other agents.

Administration of the expanded NK cells of the disclosure may be alone or in combination with IL-2 or its derivatives, immunomodulatory drugs (such as thalidomide), proteosome inhibitors (such as bortezomib) may further increase the effect of administered cells.

### EXAMPLES

The practice of the methods and compositions of the disclosure employs, unless otherwise indicated, conventional techniques of molecular biology (including recombinant techniques), cell culture, microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the methods and compositions of the disclosure. Particularly useful techniques for particular embodiments will be discussed in the sections that follow. The following materials, reagents, and methods are used for the Examples described herein.

### Isolation and Expansion of Primary Human NK Cells

Blood cones were obtained from City of Hope National Medical Center Blood Bank under the institutional review board approved protocols and NK cells were isolated by using the RosetteSep™ human NK cell enrichment cocktail (StemCell Technologies) and Ficoll-Paque (GE Healthcare). The purity of primary NK cells was confirmed with flow cytometry using anti-CD56 (Beckman Coulter, Cat #B46024) and anti-CD3 (Miltenyi Biotec, Cat #130-113-134) antibodies. The isolated NK cells were directly used for experiments or after being expanded with K562 feeder cells expressing mbIL-21 and 4-1BBL (APC K562) with or without co-expressing Tyro3 (APC K562^{Tyro3}) in the presence of IL-2 (50 IU/ml, NIH). Prior to use, the K562 feeder cells were inactivated by mitomycin (10 ug/ml) for 2 hours.

### Cell Lines

K562 cell lines were purchased from the American Type Culture Collection (ATCC). Molm-13 and U937 cells were purchased from Leibniz Institute DSMZ. The GP2-293 packaging cell line was purchased from Takara Bio. K562, Molm-13 and Jurkat cell lines were cultured in RPMI with 10% heat-inactivated FBS (Sigma-Aldrich). The GP2-293 was cultured in DMEM supplemented with 1% GlutaMax and 10% FBS. All cells were incubated at 37° C. in a 5% CO₂ humidified incubator. Cell morphology and growth characteristics were monitored during the study and compared with published reports to ensure their authenticity. All cell lines were routinely tested for the absence of mycoplasma using the MycoAlert Mycoplasma Detection Kit from Lonza. All cell lines used in experiments were cultured for less than 10 passages.

### Plasmids, Virus Production and Gene Transduction

To generate retrovirus, the GP2-293 cells were cultured till a confluency of 70-80% and then transfected with the pCIR retrovirus vector expressing Tyro3, Tyro3 fusion EGFP, ED-Tyro3, or GFP-empty vector (GFP-EV) plasmids with an envelope plasmid RD114 or RD114TR by using Lipofectamine 3000 Reagent (Thermo Fisher Scientific). The plasmids were constructed by a retroviral construction system using pCIR retrovirus vector (CytoImmune Therapeutics, CA). For retroviral transduction, coating plates with RetroNectin (Takara Bio) was performed according to a manufacturer protocol.

The various constructs used in the experiments described in the Examples have the following sequences:

Tyro3 DNA (SEQ ID NO: 1):
atggcgctgaggcggagcatggggcggccggggctcccgc
cgctgccgctgccgccgccaccgcggctcgggctgctgct
ggcggctctggcttctctgctgctcccggagtccgccgcc
gcaggtctgaagctcatgggagccccggtgaagctgacag
tgtctcaggggcagccggtgaagctcaactgcagtgtgga
ggggatggaggagcctgacatccagtgggtgaaggatggg
gctgtggtccagaacttggaccagttgtacatcccagtca
gcgagcagcactggatcggcttcctcagcctgaagtcagt
ggagcgctctgacgccggccggtactggtgccaggtggag
gatgggggtgaaaccgagatctcccagccagtgtggctca
cggtagaaggtgtgccatttttcacagtggagccaaaaga
tctggcagtgccacccaatgcccctttccaactgtcttgt
gaggctgtgggtccccctgaacctgttaccattgtctggt
ggagaggaactacgaagatcgggggacccgctccctctcc
atctgttttaaatgtaacaggggtgacccagagcaccatg
ttttcctgtgaagctcacaacctaaaaggcctggcctctt
ctcgcacagccactgttcaccttcaagcactgcctgcagc
ccccttcaacatcaccgtgacaaagctttccagcagcaac
gctagtgtggcctggatgccaggtgctgatggccgagctc
tgctacagtcctgtacagttcaggtgacacaggccccagg
aggctgggaagtcctggctgttgtggtccctgtgcccccc
tttacctgcctgctccgggacctggtgcctgccaccaact
acagcctcagggtgcgctgtgccaatgccttggggccctc
tccctatgctgactgggtgccctttcagaccaagggtcta
gccccagccagcgctccccaaaacctccatgccatccgca
cagattcaggcctcatcttggagtgggaagaagtgatccc
cgaggcccctttggaaggccccctgggaccctacaaactg
tcctgggttcaagacaatggaacccaggatgagctgacag
tggaggggaccagggccaatttgacaggctgggatcccca
aaaggacctgatcgtacgtgtgtgcgtctccaatgcagtt
ggctgtggaccctggagtcagccactggtggtctcttctc
atgaccgtgcaggccagcagggccctcctcacagccgcac
atoctgggtacctgtggtccttggtgtgctaacggccctg
gtgacggctgctgccctggccctcatcctgcttcgaaaga
gacggaaagagacgcggtttgggcaagcctttgacagtgt
catggcccggggagagccagccgttcacttccgggcagcc
cggtccttcaatcgagaaaggcccgagcgcatcgaggcca
cattggacagcttgggcatcagcgatgaactaaaggaaaa
actggaggatgtgctcatcccagagcagcagttcaccctg
ggccggatgttgggcaaaggagagtttggttcagtgcggg
aggcccagctgaagcaagaggatggctcctttgtgaaagt
ggctgtgaagatgctgaaagctgacatcattgcctcaagc
gacattgaagagttcctcagggaagcagcttgcatgaagg
agtttgaccatccacacgtggccaaacttgttggggtaag
cctccggagcagggctaaaggccgtctccccatccccatg
gtcatcttgcccttcatgaagcatggggacctgcatgcct
tcctgctegcctcccggattggggagaacccctttaacct
acccctccagaccctgatccggttcatggtggacattgcc
tgcggcatggagtacctgagctctcggaacttcatccacc
gagacctggctgctcggaattgcatgctggcagaggacat
gacagtgtgtgtggctgacttcggactctcccggaagatc
tacagtggggactactatcgtcaaggctgtgcctccaaac
tgcctgtcaagtggctggccctggagagcctggccgacaa
cctgtatactgtgcagagtgacgtgtgggcgttcggggtg
accatgtgggagatcatgacacgtgggcagacgccatatg
ctggcatcgaaaacgctgagatttacaactacctcattgg
cgggaaccgcctgaaacagcctccggagtgtatggaggac
gtgtatgatctcatgtaccagtgctggagtgctgacccca
agcagcgcccgagctttacttgtctgcgaatggaactgga
gaacatcttgggccagctgtctgtgctatctgccagccag
gaccccttatacatcaacatcgagagagctgaggagccca
ctgcgggaggcagcctggagctacctggcagggatcagcc
ctacagtggggctggggatggcagtggcatgggggcagtg
ggtggcactcccagtgactgtcggtacatactcacccccg
gagggctggctgagcagccagggcaggcagagcaccagcc
agagagtcccctcaatgagacacagaggcttttgctgctg
cagcaagggctactgccacacagtagctgttga
Tyro3 protein (SEQ ID NO: 2)
(UniProtKB ID Q06418):
MALRRSMGRPGLPPLPLPPPPRLGLLLAALASLLLPESAA
AGLKLMGAPVKLTVSQGQPVKLNCSVEGMEEPDIQWVKDG
AVVQNLDQLYIPVSEQHWIGFLSLKSVERSDAGRYWCQVE
DGGETEISQPVWLTVEGVPFFTVEPKDLAVPPNAPFQLSC
EAVGPPEPVTIVWWRGTTKIGGPAPSPSVLNVTGVTQSTM
FSCEAHNLKGLASSRTATVHLQALPAAPFNITVTKLSSSN
ASVAWMPGADGRALLQSCTVQVTQAPGGWEVLAVVVPVPP
FTCLLRDLVPATNYSLRVRCANALGPSPYADWVPFQTKGL
APASAPQNLHAIRTDSGLILEWEEVIPEAPLEGPLGPYKL
SWVQDNGTQDELTVEGTRANLTGWDPQKDLIVRVCVSNAV
GCGPWSQPLVVSSHDRAGQQGPPHSRTSWVPVVLGVLTAL
VTAAALALILLRKRRKETRFGQAFDSVMARGEPAVHFRA
ARSFNRERPERIEATLDSLGISDELKEKLEDVLIPEQQFT
LGRMLGKGEFGSVREAQLKQEDGSFVKVAVKMLKADIIAS
SDIEEFLREAACMKEFDHPHVAKLVGVSLRSRAKGRLPIP
MVILPFMKHGDLHAFLLASRIGENPFNLPLQTLIRFMVDI
ACGMEYLSSRNFIHRDLAARNCMLAEDMTVCVADFGLSRK
IYSGDYYRQGCASKLPVKWLALESLADNLYTVQSDVWAFG
VTMWEIMTRGQTPYAGIENAEIYNYLIGGNRLKQPPECME
DVYDLMYQCWSADPKQRPSFTCLRMELENILGQLSVLSAS
QDPLYINIERAEEPTAGGSLELPGRDQPYSGAGDGSGMGA
VGGTPSDCRYILTPGGLAEQPGQAEHQPESPLNETQRLLL
LQQGLLPHSSC
Tyro3-EGFP fusion DNA sequence
(SEQ ID NO: 3):
atggcgctgaggcggagcatggggcggccggggctcccgc
cgctgccgctgccgccgccaccgcggctcgggctgctgct
ggcggctctggcttctctgctgctcccggagtccgccgcc
gcaggtctgaagctcatgggagccccggtgaagctgacag
tgtctcaggggcagccggtgaagctcaactgcagtgtgga
ggggatggaggagcctgacatccagtgggtgaaggatggg
gctgtggtccagaacttggaccagttgtacatcccagtca
gcgagcagcactggatcggcttcctcagcctgaagtcagt
ggagcgctctgacgccggccggtactggtgccaggtggag
gatgggggtgaaaccgagatctcccagccagtgtggctca
cggtagaaggtgtgccatttttcacagtggagccaaaaga
tctggcagtgccacccaatgcccctttccaactgtcttgt
gaggctgtgggtccccctgaacctgttaccattgtctggt
ggagaggaactacgaagatcgggggacccgctccctctcc
atctgttttaaatgtaacaggggtgacccagagcaccatg
ttttcctgtgaagctcacaacctaaaaggcctggcctctt
ctcgcacagccactgttcaccttcaagcactgcctgcagc
ccccttcaacatcaccgtgacaaagctttccagcagcaac
gctagtgtggcctggatgccaggtgctgatggccgagctc
tgctacagtcctgtacagttcaggtgacacaggccccagg
aggctgggaagtcctggctgttgtggtccctgtgcccccc
tttacctgcctgctccgggacctggtgcctgccaccaact
acagcctcagggtgcgctgtgccaatgccttggggccctc
tccctatgctgactgggtgccctttcagaccaagggtcta
gccccagccagcgctccccaaaacctccatgccatccgca
cagattcaggcctcatcttggagtgggaagaagtgatccc
cgaggcccctttggaaggccccctgggaccctacaaactg
tcctgggttcaagacaatggaacccaggatgagctgacag
tggaggggaccagggccaatttgacaggctgggatcccca
aaaggacctgatcgtacgtgtgtgcgtctccaatgcagtt
ggctgtggaccctggagtcagccactggtggtctcttctc
atgaccgtgcaggccagcagggccctcctcacagccgcac
atcctgggtacctgtggtccttggtgtgctaacggccctg
gtgacggctgctgccctggccctcatcctgcttcgaaaga
gacggaaagagacgcggtttgggcaagcctttgacagtgt
catggcccggggagagccagccgttcacttccgggcagcc
cggtccttcaatcgagaaaggcccgagcgcatcgaggcca
cattggacagcttgggcatcagcgatgaactaaaggaaaa
actggaggatgtgctcatcccagagcagcagttcaccctg
ggccggatgttgggcaaaggagagtttggttcagtgcggg
aggcccagctgaagcaagaggatggctcctttgtgaaagt
ggctgtgaagatgctgaaagctgacatcattgcctcaagc
gacattgaagagttcctcagggaagcagcttgcatgaagg
agtttgaccatccacacgtggccaaacttgttggggtaag
cctccggagcagggctaaaggccgtctccccatccccatg
gtcatcttgcccttcatgaagcatggggacctgcatgcct
tcctgctcgcctcccggattggggagaacccctttaacct
acccctccagaccctgatccggttcatggtggacattgcc
tgcggcatggagtacctgagctctcggaacttcatccacc
gagacctggctgctcggaattgcatgctggcagaggacat
gacagtgtgtgtggctgacttcggactctcccggaagatc
tacagtggggactactatcgtcaaggctgtgcctccaaac
tgcctgtcaagtggctggccctggagagcctggccgacaa
cctgtatactgtgcagagtgacgtgtgggcgttcggggtg
accatgtgggagatcatgacacgtgggcagacgccatatg
ctggcatcgaaaacgctgagatttacaactacctcattgg
cgggaaccgcctgaaacagcctccggagtgtatggaggac
gtgtatgatctcatgtaccagtgctggagtgctgacccca
agcagcgcccgagctttacttgtctgcgaatggaactgga
gaacatcttgggccagctgtctgtgctatctgccagccag
gaccccttatacatcaacatcgagagagctgaggagccca
ctgcgggaggcagcctggagctacctggcagggatcagcc
ctacagtggggctggggatggcagtggcatgggggcagtg
ggtggcactcccagtgactgtcggtacatactcacccccg
gagggctggctgagcagccagggcaggcagagcaccagcc
agagagtcccctcaatgagacacagaggcttttgctgctg
cagcaagggctactgccacacagtagctgtggaggaggag
gaagtgcggccgccatggtgagcaagggcgaggagctgtt
caccggggtggtgcccatcctggtcgagctggacggcgac
gtaaacggccacaagttcagcgtgtccggcgagggcgagg
gcgatgccacctacggcaagctgaccctgaagttcatctg
caccaccggcaagctgcccgtgccctggcccaccctcgtg
accaccctgacctacggcgtgcagtgcttcagccgctacc
ccgaccacatgaagcagcacgacttcttcaagtccgccat
gcccgaaggctacgtccaggagcgcaccatcttcttcaag
gacgacggcaactacaagacccgcgccgaggtgaagttcg
agggcgacaccctggtgaaccgcatcgagctgaagggcat
cgacttcaaggaggacggcaacatcctggggcacaagctg
gagtacaactacaacagccacaacgtctatatcatggccg
acaagcagaagaacggcatcaaggtgaacttcaagatccg
ccacaacatcgaggacggcagcgtgcagctcgccgaccac
taccagcagaacacccccatcggcgacggccccgtgctgc
tgcccgacaaccactacctgagcacccagtccgccctgag
caaagaccccaacgagaagcgcgatcacatggtcctgctg
gagttcgtgaccgccgccgggatcactctcggcatggacg
agctgtacaagtaa
Tyro3-EGFP protein sequence
(SEQ ID NO: 4):
MALRRSMGRPGLPPLPLPPPPRLGLLLAALASLLLPESAA
AGLKLMGAPVKLTVSQGQPVKLNCSVEGMEEPDIQWVKDG
AVVQNLDQLYIPVSEQHWIGFLSLKSVERSDAGRYWCQVE
DGGETEISQPVWLTVEGVPFFTVEPKDLAVPPNAPFQLSC
EAVGPPEPVTIVWWRGTTKIGGPAPSPSVLNVTGVTQSTM
FSCEAHNLKGLASSRTATVHLQALPAAPFNITVTKLSSSN
ASVAWMPGADGRALLQSCTVQVTQAPGGWEVLAVVVPVPP
FTCLLRDLVPATNYSLRVRCANALGPSPYADWVPFQTKGL
APASAPQNLHAIRTDSGLILEWEEVIPEAPLEGPLGPYKL
SWVQDNGTQDELTVEGTRANLTGWDPQKDLIVRVCVSNAV
GCGPWSQPLVVSSHDRAGQQGPPHSRTSWVPVVLGVLTAL
VTAAALALILLRKRRKETRFGQAFDSVMARGEPAVHFRA
ARSFNRERPERIEATLDSLGISDELKEKLEDVLIPEQQFT
LGRMLGKGEFGSVREAQLKQEDGSFVKVAVKMLKADIIAS
SDIEEFLREAACMKEFDHPHVAKLVGVSLRSRAKGRLPIP
MVILPFMKHGDLHAFLLASRIGENPFNLPLQTLIRFMVDI
ACGMEYLSSRNFIHRDLAARNCMLAEDMTVCVADFGLSRK
IYSGDYYRQGCASKLPVKWLALESLADNLYTVQSDVWAFG
VTMWEIMTRGQTPYAGIENAEIYNYLIGGNRLKQPPECME
DVYDLMYQCWSADPKQRPSFTCLRMELENILGQLSVLSAS
QDPLYINIERAEEPTAGGSLELPGRDQPYSGAGDGSGMGA
VGGTPSDCRYILTPGGLAEQPGQAEHQPESPLNETQRLLL
LQQGLLPHSSCGGGGSAAAMVSKGEELFTGVVPILVELDG
DVNGHKFSVSGEGEGDATYGKLTLKFICTTGKLPVPWPTL
VTTLTYGVQCFSRYPDHMKQHDFFKSAMPEGYVQERTIFF
KDDGNYKTRAEVKFEGDTLVNRIELKGIDFKEDGNILGHK
LEYNYNSHNVYIMADKQKNGIKVNFKIRHNIEDGSVQLAD
HYQQNTPIGDGPVLLPDNHYLSTQSALSKDPNEKRDHMVL
LEFVTAAGITLGMDELYK-
ED-Tyro3 DNA sequence
(SEQ ID NO: 5):
atggcgctgaggcggagcatggggcggccggggctcccgc
cgctgccgctgccgccgccaccgcggctcgggctgctgct
ggcggctctggcttctctgctgctcccggagtccgccgcc
gcaggtctgaagctcatgggagccccggtgaagctgacag
tgtctcaggggcagccggtgaagctcaactgcagtgtgga
ggggatggaggagcctgacatccagtgggtgaaggatggg
gctgtggtccagaacttggaccagttgtacatcccagtca
gcgagcagcactggatcggcttcctcagcctgaagtcagt
ggagcgctctgacgccggccggtactggtgccaggtggag
gatgggggtgaaaccgagatctcccagccagtgtggctca
cggtagaaggtgtgccatttttcacagtggagccaaaaga
tctggcagtgccacccaatgcccctttccaactgtcttgt
gaggctgtgggtccccctgaacctgttaccattgtctggt
ggagaggaactacgaagategggggacccgctccctctcc
atctgttttaaatgtaacaggggtgacccagagcaccatg
ttttcctgtgaagctcacaacctaaaaggcctggcctctt
ctcgcacagccactgttcaccttcaagcactgcctgcagc
ccccttcaacatcaccgtgacaaagctttccagcagcaac
gctagtgtggcctggatgccaggtgctgatggccgagctc
tgctacagtcctgtacagttcaggtgacacaggccccagg
aggctgggaagtcctggctgttgtggtccctgtgcccccc
tttacctgcctgctccgggacctggtgcctgccaccaact
acagcctcagggtgcgctgtgccaatgccttggggccctc
tccctatgctgactgggtgccctttcagaccaagggtcta
gccccagccagcgctccccaaaacctccatgccatccgca
cagattcaggcctcatcttggagtgggaagaagtgatccc
cgaggcccctttggaaggccccctgggaccctacaaactg
tcctgggttcaagacaatggaacccaggatgagctgacag
tggaggggaccagggccaatttgacaggctgggatcccca
aaaggacctgatcgtacgtgtgtgcgtctccaatgcagtt
ggctgtggaccctggagtcagccactggtggtctcttctc
atgaccgtgcaggccagcagggccctcctcacagccgcac
atcctgg
ED-Tyro3 protein sequence
(SEQ ID NO: 6):
MALRRSMGRPGLPPLPLPPPPRLGLLLAALASLLLPESAA
AGLKLMGAPVKLTVSQGQPVKLNCSVEGMEEPDIQWVKDG
AVVQNLDQLYIPVSEQHWIGFLSLKSVERSDAGRYWCQVE
DGGETEISQPVWLTVEGVPFFTVEPKDLAVPPNAPFQLSC
EAVGPPEPVTIVWWRGTTKIGGPAPSPSVLNVTGVTQSTM
FSCEAHNLKGLASSRTATVHLQALPAAPFNITVTKLSSSN
ASVAWMPGADGRALLQSCTVQVTQAPGGWEVLAVVVPVPP
FTCLLRDLVPATNYSLRVRCANALGPSPYADWVPFQTKGL
APASAPQNLHAIRTDSGLILEWEEVIPEAPLEGPLGPYKL
SWVQDNGTQDELTVEGTRANLTGWDPQKDLIVRVCVSNAV
GCGPWSQPLVVSSHDRAGQQGPPHSRTSW
PDGFRB transmembrane domain DNA sequence
(SEQ ID NO: 7):
gctgtgggccaggacacgcaggaggtcatcgtggtgccac
actccttgccctttaaggtggtggtgatctcagccatcct
ggccctggtggtgctcaccatcatctcccttatcatcctc
atcatgctttggcagaagaagccacgt
PDGFRB transmembrane domain protein
sequence (SEQ ID NO: 8):
AVGQDTQEVIVVPHSLPFKVVVISAILALVVLTIISLIIL
IMLWQKKPR

The protein and nucleic acid sequences of membrane bound IL-21 and 4-1BBL are as follows: IL-21 protein sequence: UniProtKB ID—Q9HBE4 (SEQ ID NO:23). IL-21 nucleic acid sequence: NM_001207006.3 (SEQ ID NO:24). 4-1BBL (TNF Superfamily Member 9) protein sequence: UniProtKB/Swiss-Prot: P41273 (SEQ ID NO:25). 4-1BBL nucleic acid sequence: NM_003811.4 (SEQ ID NO:26).

In some embodiments, the Tyro3 sequence used in the cell lines is amino acids 41-890 of SEQ ID NO:2. In some embodiments, the membrane bound IL21 sequence used in the cell lines is amino acids 25-162 of SEQ ID NO:23. In some embodiments, the 4-1BBL sequence used in the cell lines comprises at least one of amino acids 1-28 of SEQ ID NO: 25 (the cytoplasmic domain), amino acids 29-49 of SEQ ID NO:25 (the transmembrane domain) and/or amino acids 50-254 of SEQ ID NO:25 (the extracellular domain).

The culture supernatant containing the retrovirus was harvested at 48 hrs post-transfection and filtered. The supernatants containing virus were used to infect cells lines, followed by screening with a marker such as GFP, to establish a transduced cell line. For gene transduction, RetroNectin (Takara Bio) coating plate for retroviral infection was performed according to a manufacturer protocol.

### Tyro3-Knockout Cell Line

Tyro3-knockout K562 cells were generated using CRISPR/Cas9 knockout plasmids purchased from Santa Cruz (Cat #sc-401412 and sc-401412-HDR) and used according to manufacturer's instructions. K562 cells were co-transfected with the homology-directed DNA repair (HDR) plasmid, which incorporates red fluorescent protein (RFP) for selection of cells containing a successful Cas9-induced site-specific Tyro3 knockout in genomic DNA. The cells were then single-cell sorting with Aria Fusion. The expression of Tyro3 was examined by flow cytometry.

### Reverse Transcription Polymerase Chain Reaction

Total RNA was isolated from primary NK cells with the RNeasy Mini Kit (Qiagen). Complementary DNA (cDNA) was generated by Moloney murine leukemia virus (M-MLV) reverse transcriptase (Invitrogen) and amplified by qPCR with SYBR Green (Thermo Fisher Scientific) or RT-PCR with Q5 PCR Master Mix (NEB) and gene-specific primers. Primer sequences are presented in Table 2 below. Relative amplification values were normalized to the amplification of GAPDH and/or 18S rRNA.

TABLE 2
Primer sets for PCR

| | | SEQ |
|---|---|---|
| Target | Sequence 5′ to 3′ | ID |
| IFN-γ forward | CTAATTATTCGGTAACTGACT | 9 |
| | TGA | |
| IFN-γ reverse | ACAGTTCAGCCATCACTTGGA | 10 |
| GZMB forward | TGGGGGACCCAGAGATTAAAA | 11 |
| GZMB reverse | TTTCGTCCATAGGAGACAAT | 12 |
| | GC | |
| Perforin | CGCCTACCTCAGGCTTATC | 13 |
| forward | TC | |
| Perforin | CCTCGACAGTCAGGCAGTC | 14 |
| reverse | | |
| 18S forward | TGCATGGCCGTTCTTAGTTG | 15 |
| 18S reverse | AGTTAGCATGCCAGAGTCTC | 16 |
| | GTT | |
| Tyro3- | CAGTGACTGTCGGTACATAC | 17 |
| endogenous | TC | |
| Tyro3- | CATCAAGCCAAGGCCTAAAC | 18 |
| endogenous | | |
| Tyro3- | CAGTGACTGTCGGTACATAC | 19 |
| transgene | TC | |
| Tyro3- | CTGAACTTGTGGCCGTTTAC | 20 |
| transgene | | |
| GAPDH forward | ATGGGGAAGGTGAAGGTCGGA | 21 |
| | GTCAA | |
| GAPDH reverse | CGGAGGGGCCATCCACAGT | 22 |
| | CTTCT | |

### Immunoblotting Assay

Cells were harvested and suspended in RIPA lysis buffer (Thermo Fisher Scientific) on ice for 20 minutes. Equal amount of proteins was resolved by 5-15% Criterion TGX gel (Bio-Rad) and then transferred onto a Nitrocellulose (NC) or PVDF membrane (Thermo Fisher Scientific). The membrane was incubated with a primary antibody at 4° C. overnight and an IRDye secondary antibody (Li-COR Biosciences) for 1 hr at room temperature. The immunoblots were visualized with Odyssey CLx Imager. Densitometric analysis was performed to quantify intensity of gel bands with Image J.

### 51Cr-Release Cytotoxicity Assay

⁵¹Cr cytotoxicity assay was performed as described previously (Mrozek et al., 1996). Primary NK cells were co-cultured with K562 cells and IL-2 (150 IU/ml; NIH) for 24 hrs, and then Tyro3⁻ and Tyro3⁺ NK cells were sorted with FACSAria Fusion (BD Bioscience). Purified Tyro3⁻ and Tyro3⁺ NK cells were co-cultured with ⁵¹Cr labeled K562 cells in triplicates in a 96-well U-bottom plate at multiple ET ratios for 4 hrs at 37° C. in a 5% CO₂. The supernatant was harvested from each well and transferred into 96-well Luma plate and analyzed using a Microbeta scintillation counter (Wallac, PerkinElmer).

### Degranulation Assay

Primary NK cells were co-cultured with K562 cells and IL-2 (150 IU/ml; NIH) for 24 hrs, and then NK cells were plated in a 96-well round bottom plate with anti-human CD107a antibody (BD Biosciences) and 1 mg/ml GolgiPlug (BD Biosciences) for a 4 hr incubation. Then cells were stained with anti-CD56 antibodies and analyzed by flow cytometry.

### Flow Cytometry

Cells were stained with monoclonal antibodies at room temperature for 20 minutes and washed with FACS buffer prior to analysis using a Fortessa X 20 flow cytometry (BD Biosciences). For IFN-γ intracellular flow cytometric analysis, 1 mg/ml GolgiPlug (BD Biosciences) was added for 4 hrs before cell harvest. Then cells were permeabilized and fixed using a Cytofix/Cytoperm Fixation/Permeabilization Solution Kit (BD Biosciences). Data was analyzed using Flowjo V10 software (Tree Star, Ashland, OR, USA). Information on flow antibodies is presented in Table 1 below.

### NSG Xenograft Model

NOD-SCID-IL2Rγ−/− (NSG) mice were purchased from the Jackson Laboratory and housed at the City of Hope Animal Facility. Primary human NK cells were incubated with APC K562 cells or APC K562^{Tyro3} cells, which were inactivated by mitomycin (10 ug/ml) prior to use, in the presence of IL-2 (50 IU/ml) for 4 days and then transduced with soluble IL-15 (sIL15) retrovirus for 48 h, followed by being incubated with inactivated APC K562 cells or APC K562^{Tyro3} cells in the presence of IL-2 (50 IU/ml) for another 7 days prior to being harvested and frozen for mouse injection. For in vivo studies, on day 0, mice were intravenously (i.v.) injected with 1×10⁶ K562-luciferase (K562_Luc) cells and then i.v. treated with 10×10⁶ aforementioned sIL15 NK cells expanded with APC K562 (APC K562_sIL15 NK) or APC K562^{Tyro3} (APC K562^{Tyro3} sIL15 NK). On day 1, mice were i.v. treated with the 2nd dose of these NK cells. Bioluminescence imaging was performed on days 9 and 14. All animal experiments were approved by the City of Hope Animal Care and Use Committee.

### Statistical Analysis

Two independent or paired groups were compared by student two-tailed t-tests or paired t-tests. Multiple groups were compared using one-way or two-way ANOVA. A linear mixed model or one-way ANOVA model with repeated measures was used to account for the variance-covariance structure due to repeated measures from the same subject. P values were adjusted for multiple comparisons by Tukey's or Holm-Sidak's procedure. A P value of 0.05 or less was considered statistically significant. GraphPad 9.1.0 was used for the statistical analysis in the current study.

TABLE 1
Source of antibodies used in Examples that follow

| Antibody | Conjugation | Company | Cat# |
|---|---|---|---|
| CD56 | APC | Beckman Coulter | B46024 |
| CD56 | BV421 | BD Bioscience | 562751 |
| CD56 | PE | Beckman Coulter | IM2073U |
| CD3 | VioGreen | Miltenyi Biotec | 130-113-134 |
| CD3 | FITC | BD Bioscience | 555339 |
| Tyro3 | PE | R&D | FAB859P |
| Mer | PE | R&D | FAB8912P |
| Axl | PE | R&D | FAB154P |
| CD107a | BV786 | BD Bioscience | 564349 |
| IFN-γ | BUV395 | BD Bioscience | 743032 |
| Sytox Blue | BV405 | Thermo Fisher | S34857 |
| Annexin V | APC | BD Bioscience | 550474 |
| CD69 | FITC | BD Bioscience | 557049 |
| CD25 | APC | BD Bioscience | 555434 |
| CD62L | FITC | BD Bioscience | 555543 |
| TRAIL | APC | BD Bioscience | 563642 |
| NKG2D | PE-Cy7 | BD Bioscience | 562365 |
| CD94 | FITC | Biolegend | 305504 |
| KLRG1 | AF488 | eBioscience | 53-9488-42 |
| NKG2A | FITC | Miltenyi Biotec | 130-113-563 |
| Tim-3 | APC-Cy7 | Biolegend | 345026 |
| TIGIT | BV421 | BD Bioscience | 565270 |
| PD-L1 | Biotin | GenScript | A01950 |
| CD11b | PE-Cy7 | BD Bioscience | 557743 |
| 41BBL | PE | BD Bioscience | 559446 |
| mIL-21 | AF647 | BD Bioscience | 560493 |
| Anti-Biotin | FITC | Miltenyi Biotec | 130-113-852 |

### Example 1: Tyro3 Expression on Primary Human NK Cells Rapidly Induced by Tyro3⁺ Tumor Cells

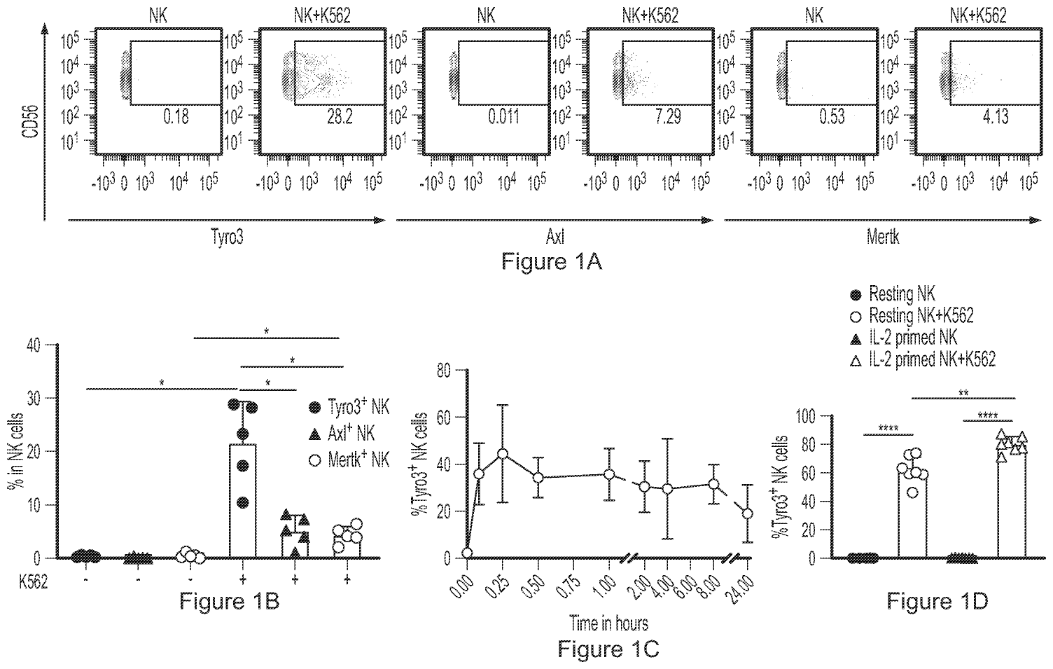

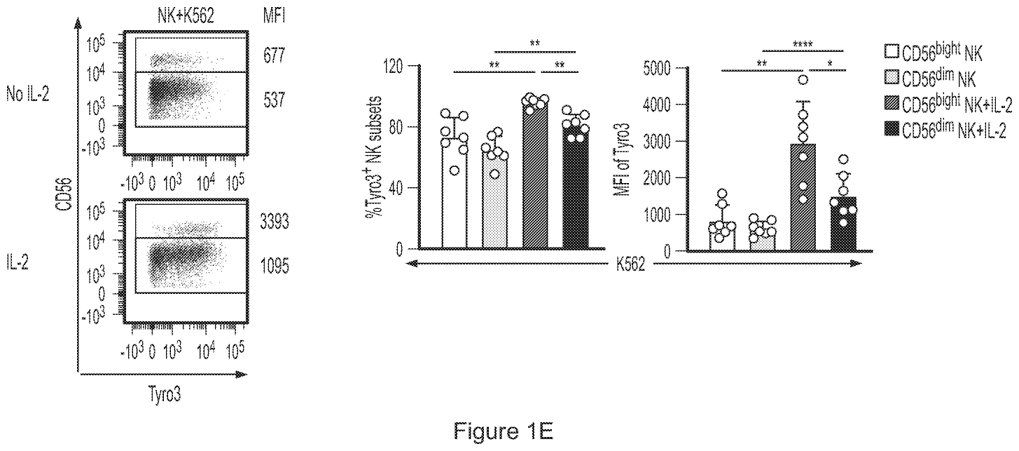

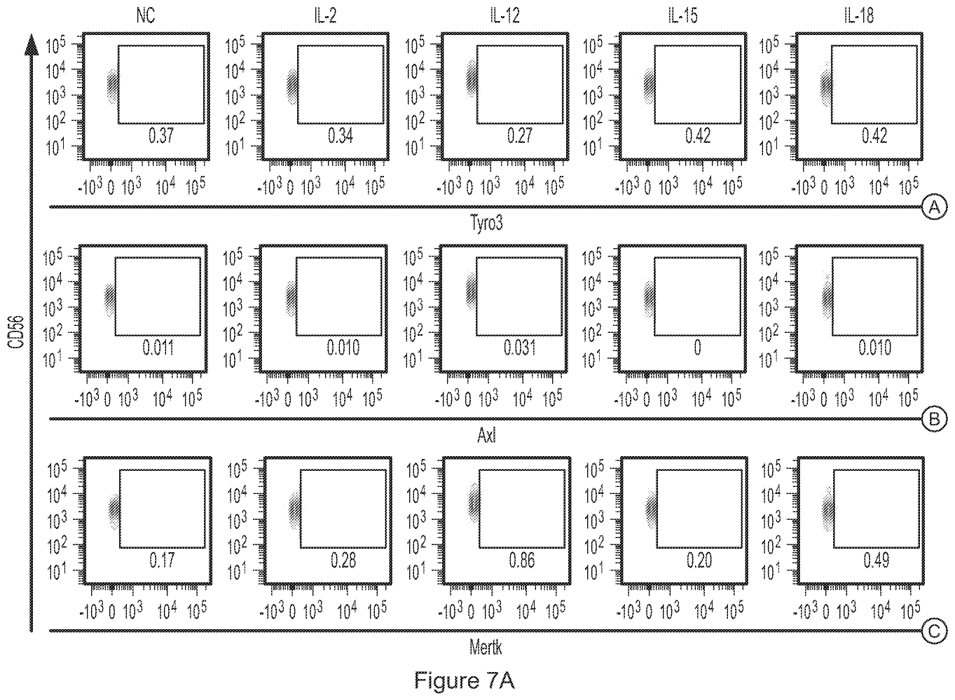

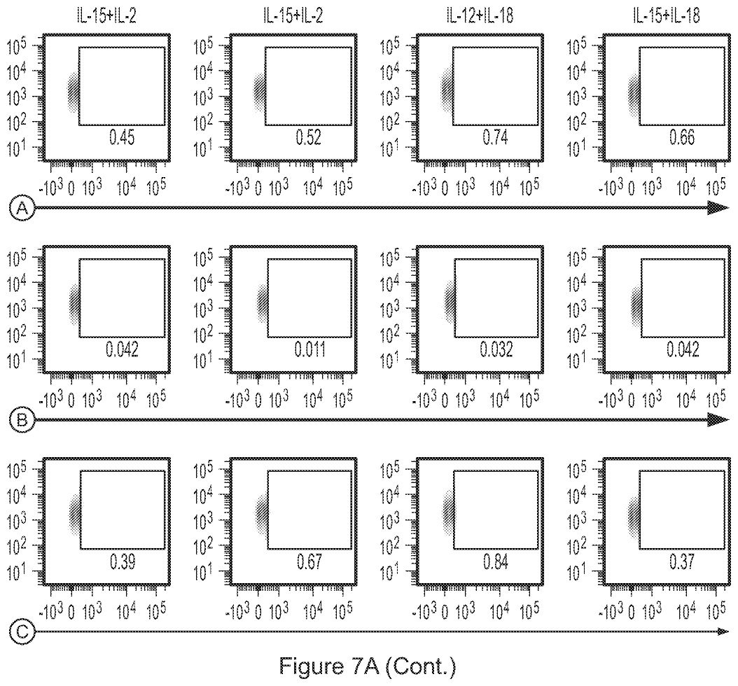

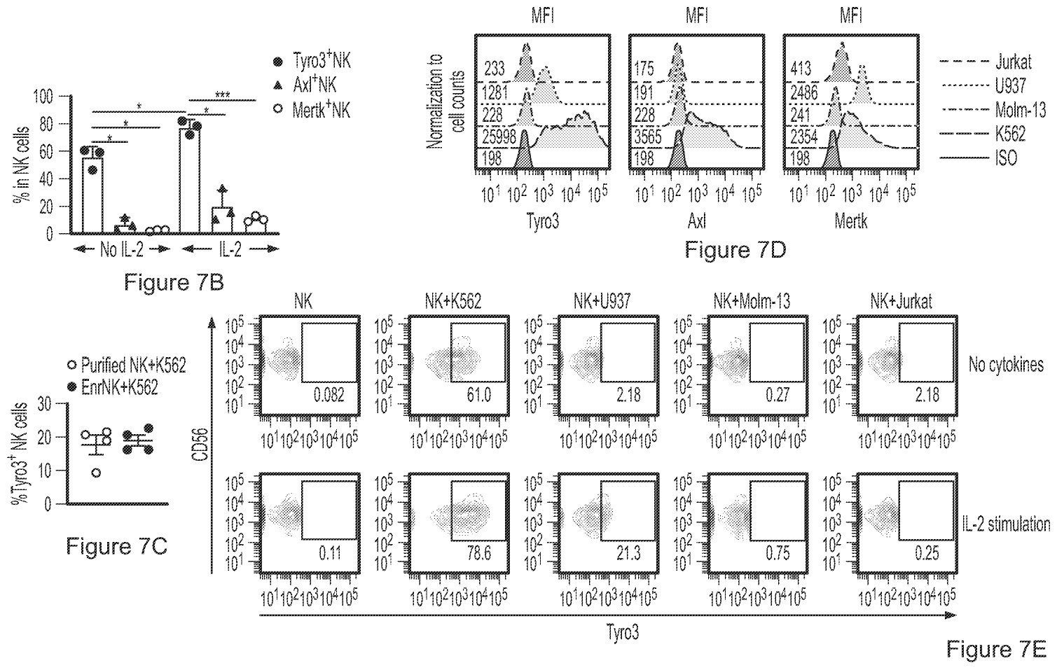

To investigate whether activated NK cells express TAM family receptors, human primary NK cells were primed with the different cytokines (e.g., cytokine IL-2) or allowed the NK cells to encounter the K562 myeloid leukemia cell line for 24 hrs. Flow cytometry analysis showed that NK cells largely expressed Tyro3, but only minimal Axl and Mertk after encountering K562 cells (FIG. 1A). However, resting NK cells did not express any TAM family receptors on the surface and the negative expression did not change when the NK cells were primed with single or combined cytokines in the absence of K562 cells (FIG. 7A). Next, the expression of Tyro3 was measured at different time points when primary human NK cells were co-cultured with K562 cells. Tyro3 expression could be largely detected on NK cells even within 5 minutes of co-culture with K562 cells and reached a plateau after 15 min of co-incubation (FIG. 1C). The rapid detection of Tyro3 on the NK cell surface, which could occur within 5 minutes, suggests that the upregulation of Tyro3 may not depend on the gene transcription and translation process. Although IL-2 alone could not induce Tyro3 expression on NK cells (FIGS. 1D and 7A), NK cells that were first primed with IL-2, expressed more Tyro3 following incubation with K562 cells when compared to resting NK cells incubated with K562 cells but without IL-2 priming (FIGS. 1D and 7B). Interestingly, IL-2 primed CD56^{bright} NK cells express more Tyro3 than IL-2 primed CD56^{dim} NK cells after incubation with K562 cells for 1 hr (FIG. 1E). These results indicate that activated NK cells express more Tyro3 that resting NK cells after encountering K562 cells. Moreover, no difference was found in K562-induced Tyro3 expression between enriched NK cells and highly purified (>97%) NK cells (FIG. 7C).

Next, flow cytometric analysis showed that K562 and U937 cell lines have high expression levels of Tyro3, while Molm-13 and Jurkat cell lines barely express Tyro3 (FIG. 7D). After NK cells were co-cultured with the different tumor cell lines, NK cells expressed higher levels of Tyro after being incubated with K562 or U937 cells than with Molm-13 or Jurkat cells in the presence of IL-2, suggesting that Tyro3 expression levels in tumor cells correlate with Tyro3 induction levels in NK cells (FIG. 7E).

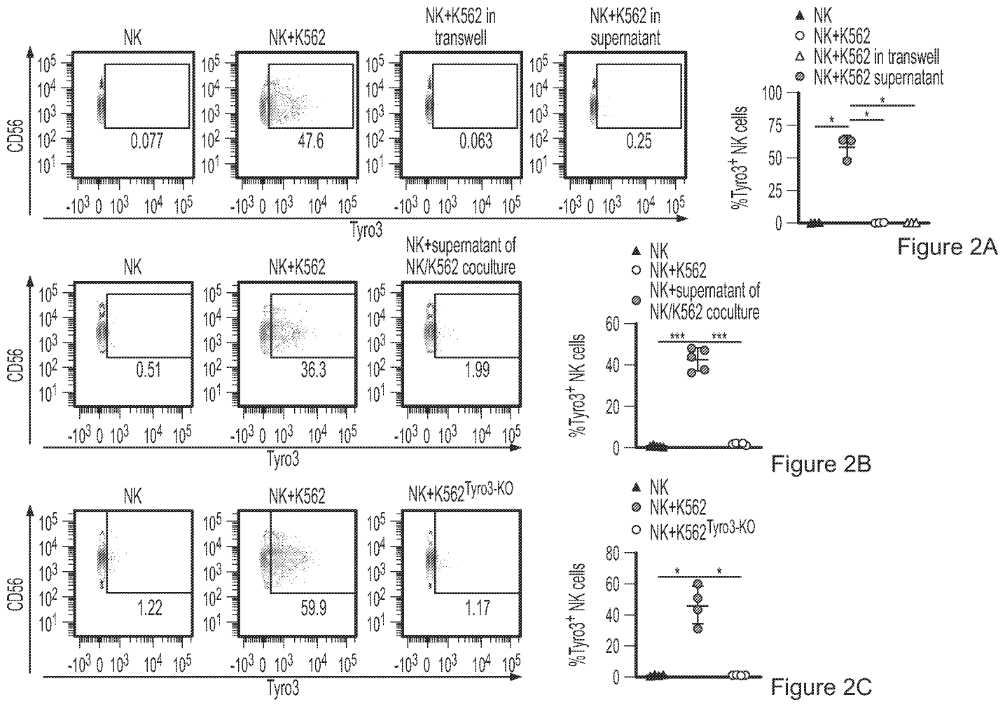

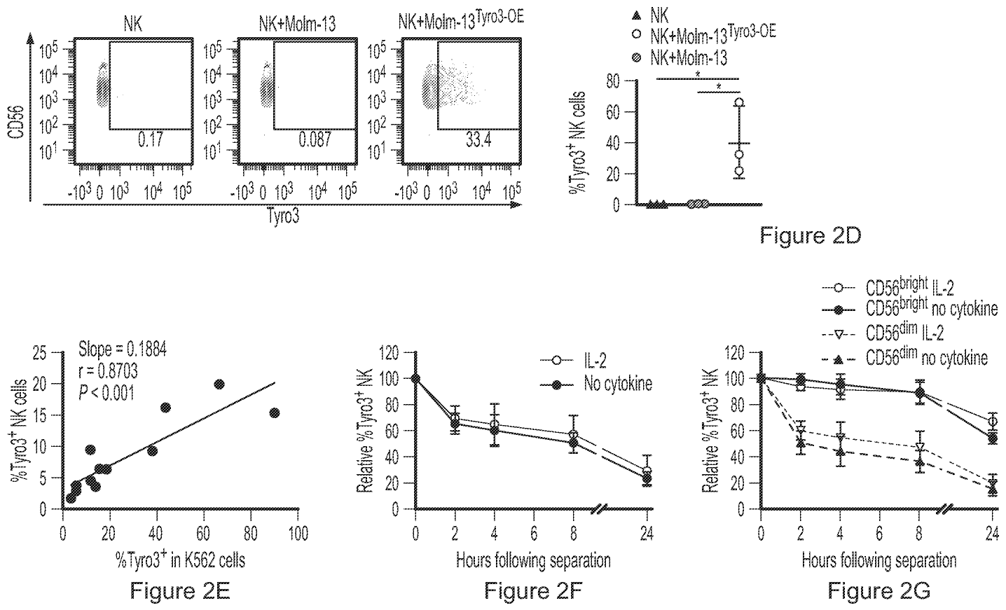

The mechanism of Tyro3 upregulation in NK cells was determined by testing whether direct cell contact was required for Tyro3 induction. K562 cells incubated in transwells could not induce Tyro3 expression on NK cells (FIG. 2A). NK cells were then cultured in the supernatants from K562 cells alone or with the supernatants from K562 cells incubated with NK cells. Neither set of conditioned media was able to induce Tyro3 expression on NK cells (FIGS. 2A-B). These data show that direct interaction between NK cells and the K562 myeloid leukemia cells is necessary to induce rapid Tyro3 expression on NK cells.

### Example 2: Tyro3 Expression on NK Cells Rapidly Induced by Tyro3⁺ Tumor Cells Requires Cell-Cell Contact

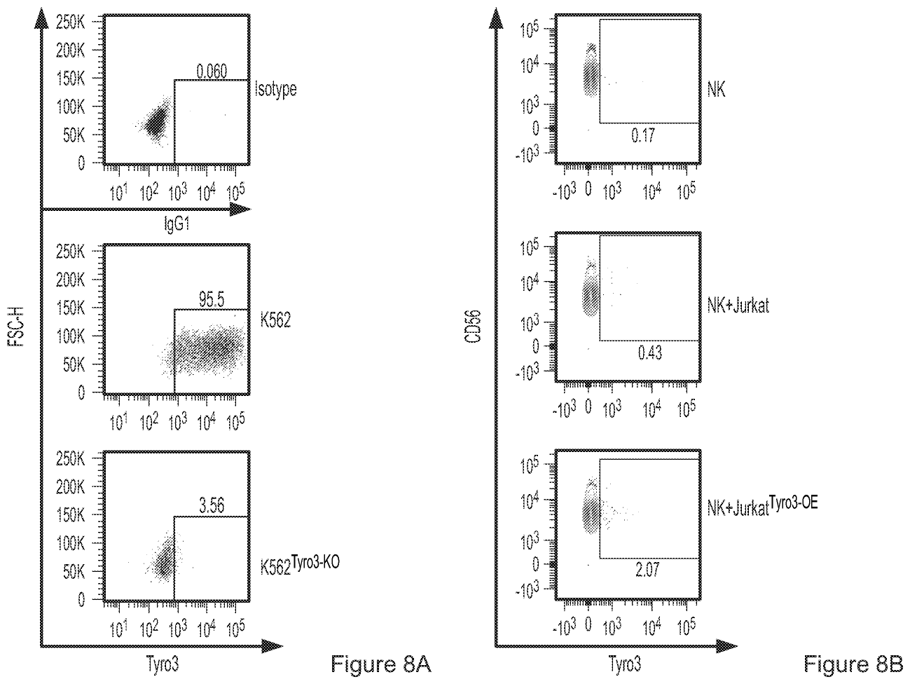

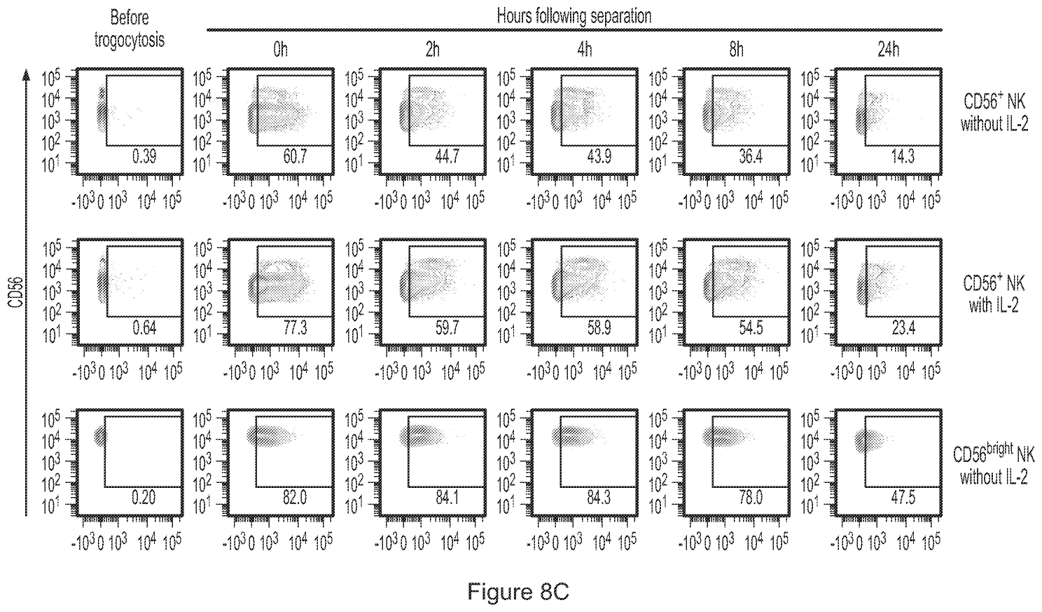

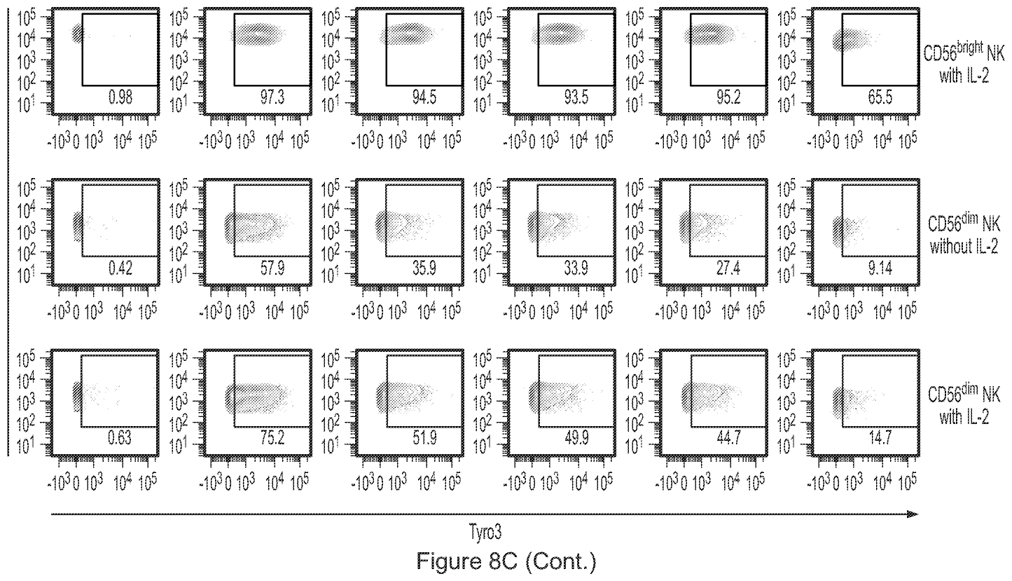

Since Tyro3 expression levels on the surface of tumor cells correlated with the level of Tyro3 increase in NK cells (FIGS. 7D AND 7E), Tyro3⁻ deficient K562 cells (K562^{Tyro3-KO}) were established by CRISPR/Cas9-mediated genome editing (FIG. 8A). The induction of Tyro3 expression on NK cells failed when being co-cultured with K562^{Tyro3-KO} cells (FIG. 2C). On the other hand, Tyro3 expression on NK cells co-cultured with Molm-13 cells overexpressing Tyro3 (Molm-13^{Tyro3-OE}) was considerably upregulated as compared to NK cells co-cultured with parental Molm-13 cells (FIG. 2D), while NK cells could not acquire Tyro3 from Jurkat cells overexpressing Tyro3 (Jurkat^{Tyro3-OE}) (FIG. 8B). Since the level of protein expression in the donor cells seemed to affect the amount of transferred protein, different K562 cell lines with different Tyro3 knockout efficiencies were generated using a CRISPR/Cas9-mediated genome editing procedure. The increasing rate of Tyro3 expression in NK cells positively correlated with the Tyro3 expression levels of K562 cells (r=0.8703, p<0.001) (FIG. 2E). Furthermore, half of the NK cells that had acquired Tyro3 from K562 cells no longer displayed it after 8 hrs when they had been purified and separated from K562 cells (FIGS. 2F and 8C), regardless of IL-2 priming, suggesting that a continuous presence of target cells is required to maintain Tyro3 expression on NK cells. Interestingly, Tyro3 acquired by CD56^{bright} NK cells showed a better persistence than that acquired by CD56^{dim} NK cells regardless of IL-2 priming (FIGS. 2G and 8C), consistent with the observation that IL-2 primed CD56^{bright} NK cells acquired more Tyro3 than CD56^{dim} cells (FIG. 1E). Collectively, these results demonstrate that induction of Tyro3 expression on NK cells by Tyro3⁺ tumor cells happens rapidly and requires cell-to-cell contact.

### Example 3: Tyro3-EGFP Fusion Protein can be Transferred from Tumor Cells to Human NK Cells via Trogocytosis

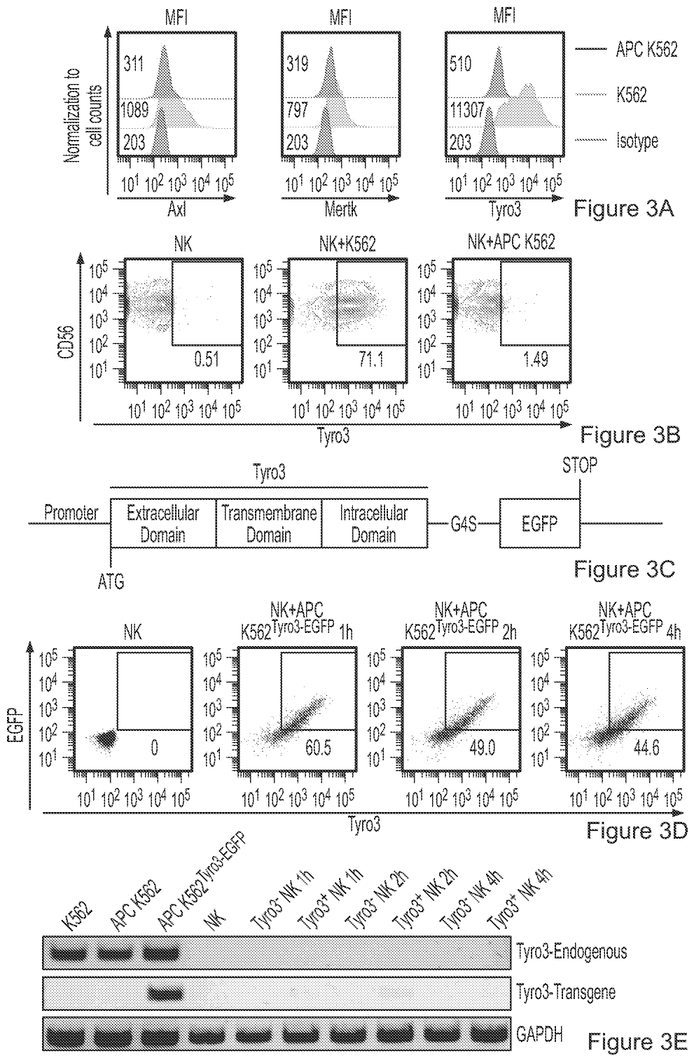

The visualization of Tyro3 transfer from tumor cells to NK cell by short-term direct contact with tumor cells was investigated. The K562 cell line expressing membrane bound IL-21 (mbIL-21) and 4-1-BBL (APC K562) is usually used to expand NK cells in vitro. Clinical grade master and working cell banks of APC K562 cells were established for NK cell expansion. Flow cytometry analysis showed APC K562 cells had absent or low protein expression levels of Tyro3, Axl or Mertk, while the parental K562 cells expressed all of these three proteins, with the highest level for Tyro3 (FIG. 3A). Consistent with this, co-incubation of primary human NK cells with APC K562 cells was not found to induce Tyro3 expression on NK cells (FIG. 3B). To test whether ectopically expressed Tyro3 can be transferred from APC K562 cells to NK cells, APC K562 cells were transduced with a Tyro3-EGFP fusion protein, in which the intracellular part of Tyro3 was followed with EGFP at the protein C terminal with a G4S (Gly-Gly-Gly-Gly-Ser) linker (SEQ ID NO:27), (APC K562^{Tyro3-EGFP}). EGFP fluorescence as well as Tyro3 extracellular domains were transferred from the APC K562^{Tyro3-EGFP} cells to primary human NK cells after co-incubation of NK cells with APC K562^{Tyro3-EGFP} cells, indicating that an entire protein instead of a part of protein, such as a shed extracellular domain, was transferred from the tumor cells to NK cells (FIG. 3D).

To show that the Tyro3 displayed on NK cells after co-incubation with tumor cells was not endogenously produced, Tyro3⁺ NK cells and Tyro3⁻ NK cells were sorted from co-culture with APC K562^{Tyro3-EGFP} cells at different time points and measured the transcription of Tyro3 by PCR. One pair of primers (SEQ ID NOs:17-18) used are specific for native Tyro3 mRNA and lack the ability to amplify Tyro3 mRNA derived from transfected APC K562^{Tyro3-EGFP} cells, while the other pair of primers (SEQ ID NOs:19-20) used are specific for transfected APC K562^{Tyro3-EGFP} cells and unable to amplify native Tyro3 mRNA. The primer sequences used are shown in Table 2 above.

After co-incubation with APC K562^{Tyro3-EGFP} cells, NK cells displayed cell-surface Tyro3 (FIG. 3D), but not Tyro3 mRNA regardless of endogenous or transduced ones (FIG. 3E). Collectively, these data show that NK cells acquire Tyro3 from the K562 myeloid leukemia cell line via a process of trogocytosis.

### Example 4: The NK Subset Acquiring Tyro3 via Trogocytosis (Tyro3⁺ NK Cells) Possesses Improved Effector Functions

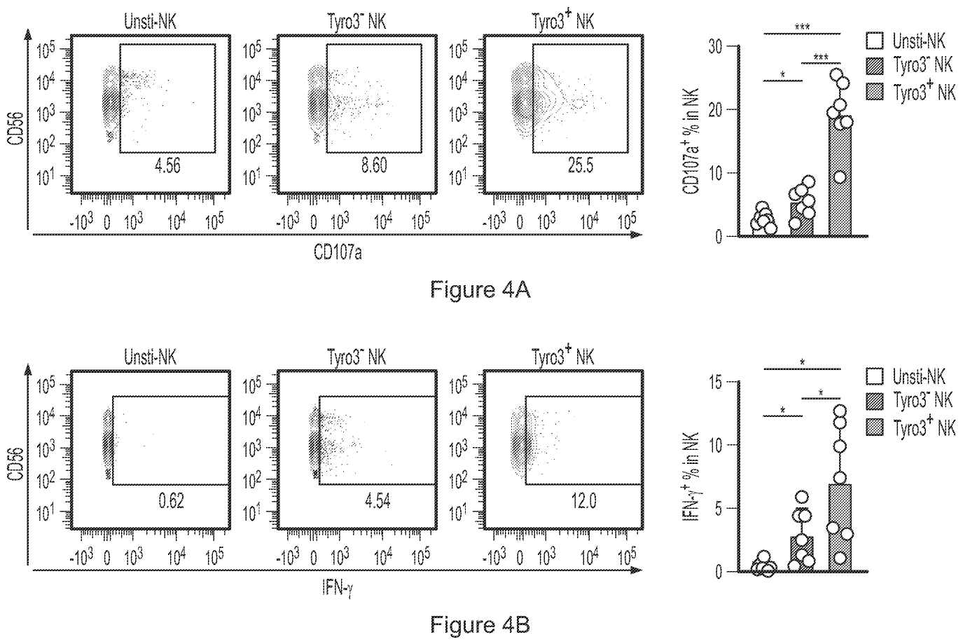

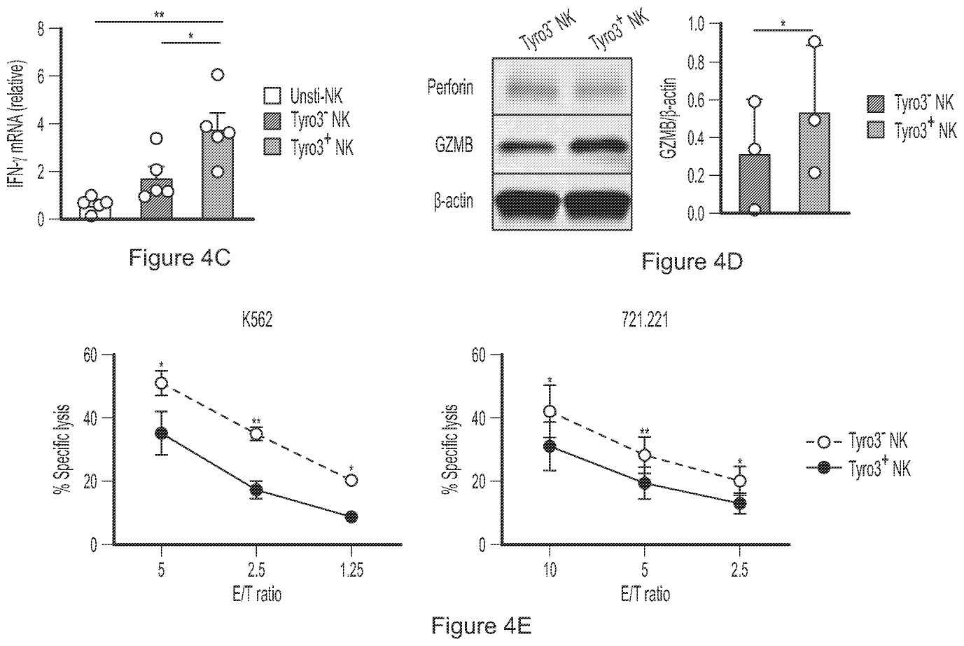

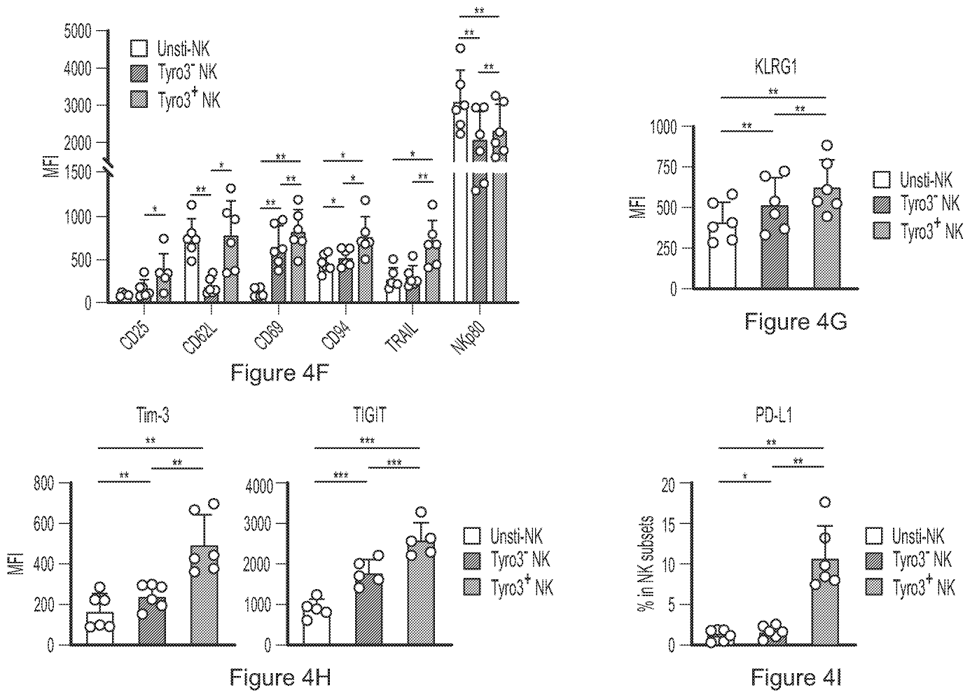

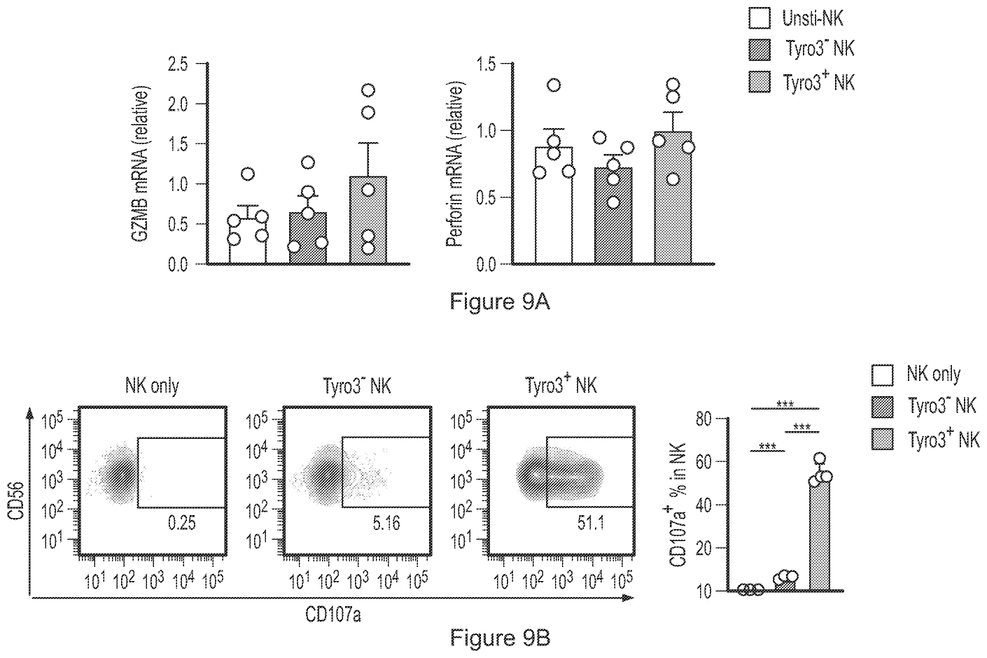

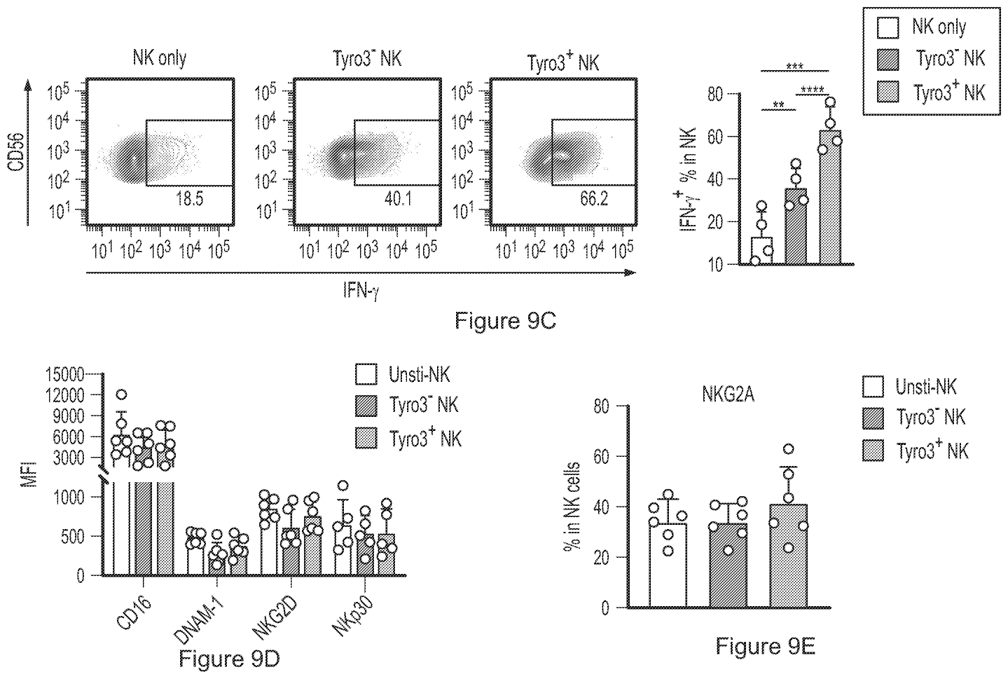

The function of Tyro3⁺ NK cells was investigated. NK cell expression of CD107a and IFN-γ are commonly used as functional markers for NK cell degranulation and cytokine production, respectively, following NK cell activation. After co-culture of NK cells with K562 cells for 24 hrs, the expression of CD107a and IFN-γ was found to be significantly increased in Tyro3⁺ NK cells compared to Tyro3⁻ NK cells (FIGS. 4A and 4B). The IFN-γ mRNA expression levels also significantly increased in the Tyro3⁺ NK cell subset compared to the Tyro3⁻ NK cell subset (FIG. 4C), while there were no significant differences in granzyme B (GZMB) and perforin mRNA expression levels between the Tyro3⁺ and Tyro3⁻ NK cell subsets (FIG. 9A). Moreover, even after co-culture of IL-2-primed NK cells with K562 cells for 4 hrs, the expression of CD107a and IFN-γ significantly increased in Tyro3⁺ NK cells (FIGS. 9B and 9C), with an obviously increased protein level of GZMB but not perforin (FIG. 4D), compared to Tyro3⁻ NK cells. with an obviously increased protein level of GZMB but not perforin (FIG. 4D), compared to Tyro3⁻ NK cells. The ⁵¹Cr release assay confirmed that the cytotoxicity level of Tyro3⁺ NK cells was significantly increased compared to Tyro3⁻ NK cells (FIG. 4E). These results further indicate that Tyro3⁺ NK cells are highly activated immune effector cells. Collectively, these data demonstrate that NK cells acquired Tyro3 upon encountering tumor cells and compared to Tyro3⁻ NK cells, Tyro3⁺ NK cells possess more robust effector functions against target cells.

### Example 5: Surface Markers Expression on Tyro3⁺ and Tyro3⁻ NK Cells

Since changes of NK cell surface receptor expression is commonly reported in cancers (Nieto-Velazquez N G, et al. Transl Oncol. 2016; 9(5):384-91; Kono K, et al. Clin Cancer Res. 1996; 2(11):1825-8; Fauriat C, et al. Leukemia. 2006; 20(4):732-3) and the above data show that the Tyro3⁺ and Tyro3⁻ NK cells may have differing functional roles, the expression of surface markers on these two distinct NK cell subsets using flow cytometry was evaluated. Co-incubation of NK cells with K562 cells led to increased expression of several activation markers, CD25, CD69, and TRAIL, which were found to be significantly increased on Tyro3⁺ NK cells compared to Tyro3⁻ NK cells and unstimulated NK cells (FIG. 4F), while the other activation receptors such as NKG2D and NKp30 did not show a significant difference in expression between Tyro3⁺ and Tyro3⁻ NK cells (FIG. 9D). Moreover, CD62L expression was maintained in Tyro3⁺ NK cells while decreased in Tyro3⁻ NK cells compared to unstimulated NK cells. Cell maturation markers CD94, NKp80, and KLRG1 showed an increase in expression in the Tyro3⁺ NK cell subset compared to the Tyro3⁻ NK cell subset or unstimulated NK cells, while the inhibitory receptor NKG2A did not show any difference in expression between the Tyro3⁺ and Tyro3⁻ NK cell subsets (FIGS. 4F-4G and 9E). Furthermore, co-incubation of NK cells with K562 cells induced the expression of the two exhaustion-related markers, Tim-3 and TIGIT, with expression levels significantly higher in Tyro3⁺ NK cells than Tyro3⁻ NK cells (FIG. 4H).

It has previously been shown that PD-L1 was induced on NK cells after encountering K562 cells, suggesting enhanced NK cell function and preventing cell exhaustion (Dong W, et al. Cancer discovery. 2019; 9(10):1422-37). The surface marker expression data show that PD-L1 expression was significantly upregulated in the Tyro3⁺ NK cell subset compared to the Tyro3⁻ NK cell subset after NK cells encountered K562 cells for 24 hrs (FIG. 4I).

### Example 6: Acquisition of Tyro3 by NK Cells from APC K562 Cells Expressing Tyro3 Enhanced NK Cells Ex Vivo Expansion

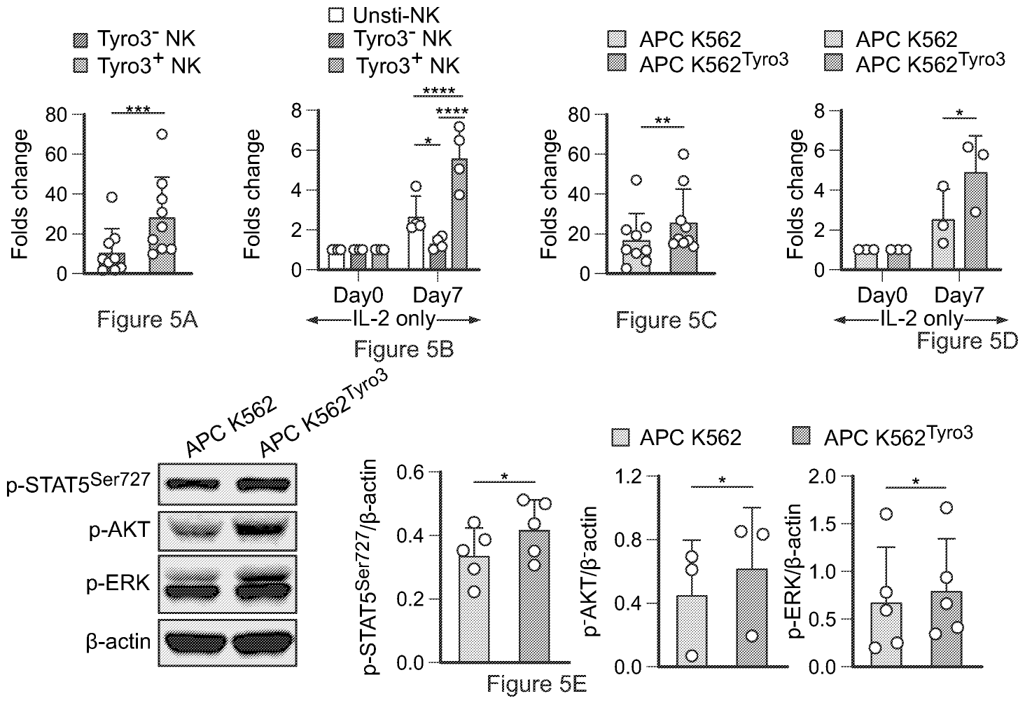

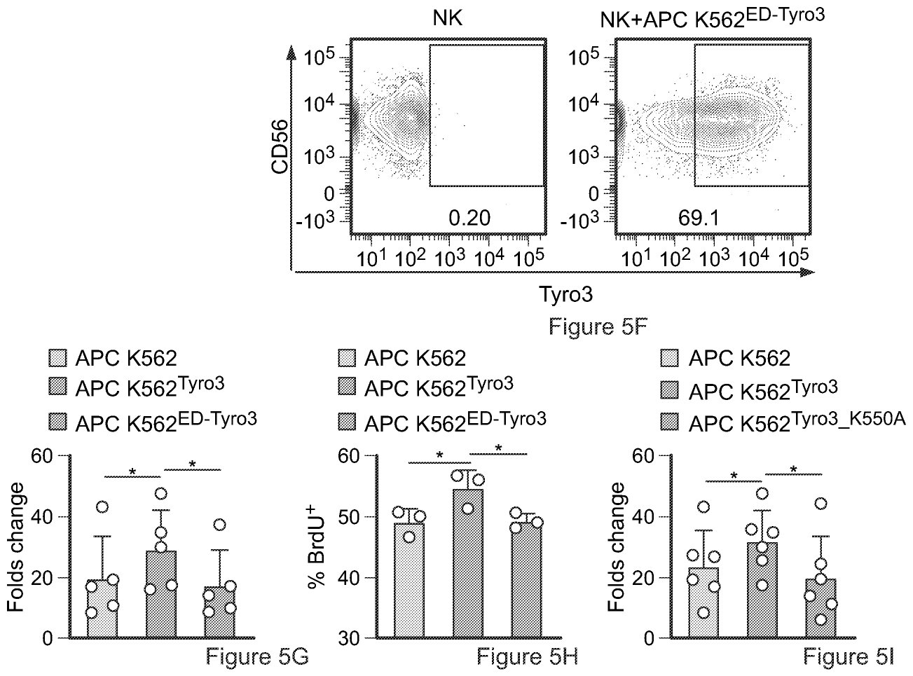

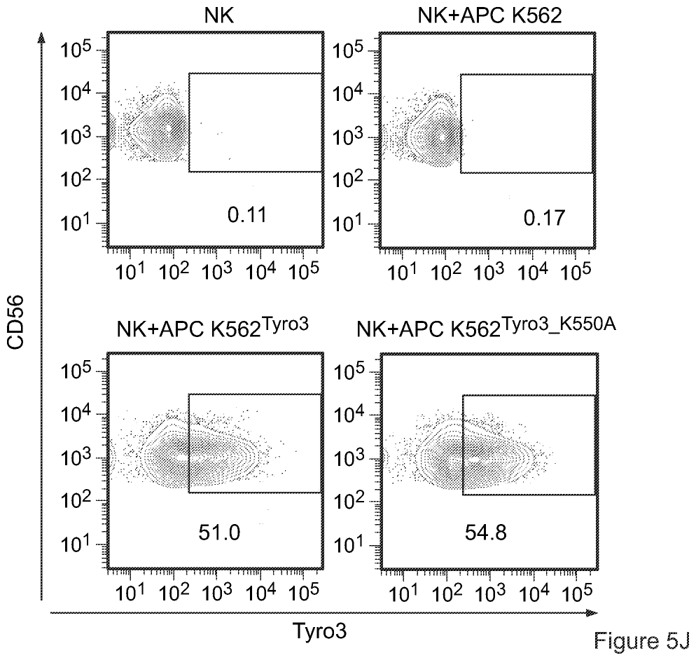

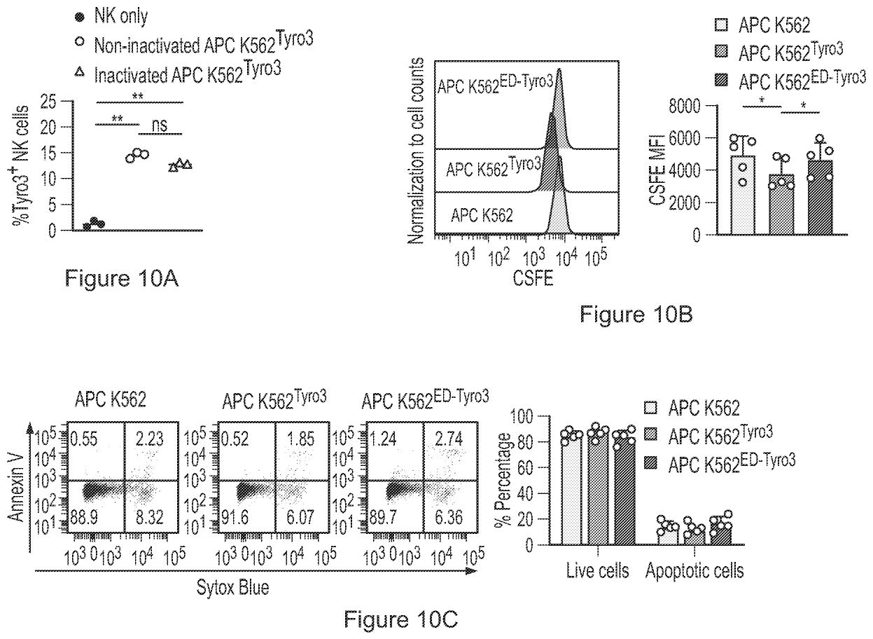

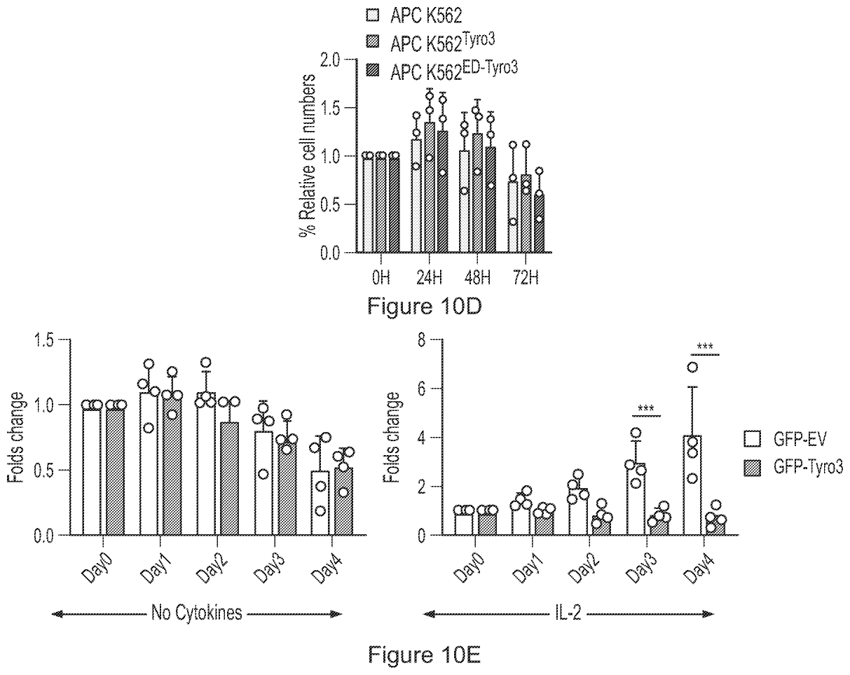

After NK cells were incubated with parental K562 cells for 24 hrs, Tyro3⁺ and Tyro3⁻ NK cells were sorted, and then expanded with APC K562 cells, which expresses 4-1BBL and mbIL-21 in the presence of IL-2 (50 IU/ml). After 7 days, the expansion folds of Tyro3⁺ NK cells were significantly higher when compared to the expansion folds of Tyro3⁻ NK cells (FIG. 5A). IL-2 (150 IU/ml) by itself also enhanced Tyro3⁺ NK cell proliferation compared with Tyro3⁻ NK cells (FIG. 5B), suggesting that Tyro3⁺ NK cells are more responsive to IL-2 than Tyro3⁻ NK cells. APC K562 express much lower Tyro3 on the surface compared to unengineered parental K562 (FIG. 3A). APC K562 cell lines that stably overexpressed full-length Tyro3 (APC K562^{Tyro3}) or the extracellular domain of Tyro3 with the PDGFRB transmembrane domain (APC K562^{ED-Tyro3}) were made by retrovirally transducing K562 cells with Tyro3 or ED-Tyro3. Since inactivated APC K562 cells are being used to expand unmodified primary human NK cells or engineered NK cells, such as CAR NK cells, for clinical use, the ability of primary human NK cells to acquire Tyro3 from APC K562 cells inactivated by mitomycin was tested. Indeed, primary human NK cells were observed to capture Tyro3 from inactivated APC K562^{Tyro3} cells, which was similar to that from the non-inactivated APC K562^{Tyro3} cells (FIG. 10A). The expansion of NK cells co-incubated with APC K562^{Tyro3} and IL-2 for 7 days was significantly higher than the expansion of NK cells co-incubated with APC K562 and IL-2 for 7 days (FIG. 5C). With IL-2 treatment, NK cells pre-incubated with APC K562^{Tyro3} proliferated significantly more than those with APC K562 (FIG. 5D).

Furthermore, NK cells were separated from tumor cells after co-incubation with APC K562 or APC K562^{Tyro3} for 1 hr, and the levels of STAT5p-AKT and p-ERK in NK cells were then evaluated by immunoblotting. The amount of STAT5, p-AKT and p-ERK in NK cells increased more with APC K562^{Tyro3} stimulation compared to APC K562 stimulation (FIG. 5E). When NK cells were co-cultured with APC K562^{ED-Tyro3} for 1 hr, the expression of Tyro3 was detected on the surface of NK cells (FIG. 5F). However, APC K562^{ED-Tyro3} did not show the same benefits on NK cell expansion as observed with APC K562^{Tyro3} (FIG. 5G). In NK cells expanded with APC K562^{Tyro3}, the percentage of BrdU incorporated into DNA (FIG. 5H) and proliferation rate (FIG. 10B) was significantly higher than in those expanded with APC K562 or APC K562^{ED-Tyro3} while there was no difference in cell apoptosis or cell survival among these three different groups of expanded NK cells (FIGS. 10C-10D). We next generated APC K562 cells that stably overexpressed kinase dead full-length Tyro3 (APC K562^{Tyro3_K550A}) with a K550A mutation (34). Similar to APC K562^{ED-Tyro3}, APC K562^{Tyro3_K550A} did not show the same benefits on NK cell expansion as observed with APC K562^{Tyro3} (FIG. 5I) while the expression of Tyro3 was detected on the surface of NK cells when NK cells were co-cultured with APC K562^{Tyro3_K550A} (FIG. 5J).

Taken together, results suggest that Tyro3 transferred to NK cells via trogocytosis may initiate signaling to control NK cell activation. Consistent with a previous report (Paolino M, et al. Nature. 2014; 507(7493):508-12), overexpression of Tyro3 in NK cells by transduction did not enhance NK cell expansion regardless of the presence or absence of IL-2 (FIG. 10E).

### Example 7: An Equal Number of NK Cells Expanded by K562 Feeder Cells and Their Counterpart Expressing Tyro3 have Comparable In Vitro and In Vivo Effector Functions

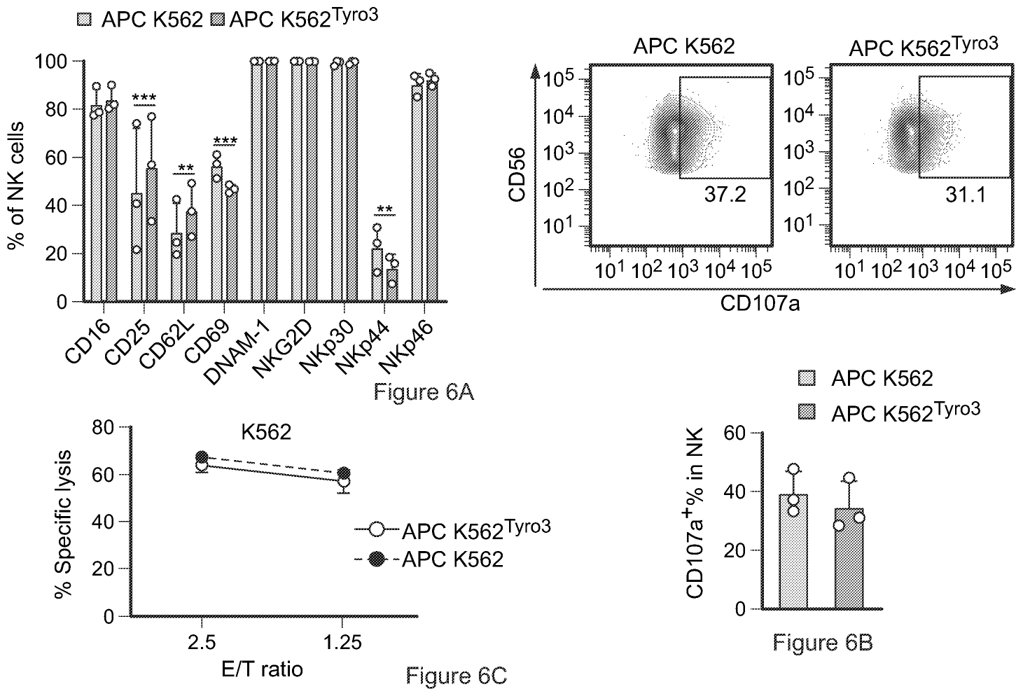

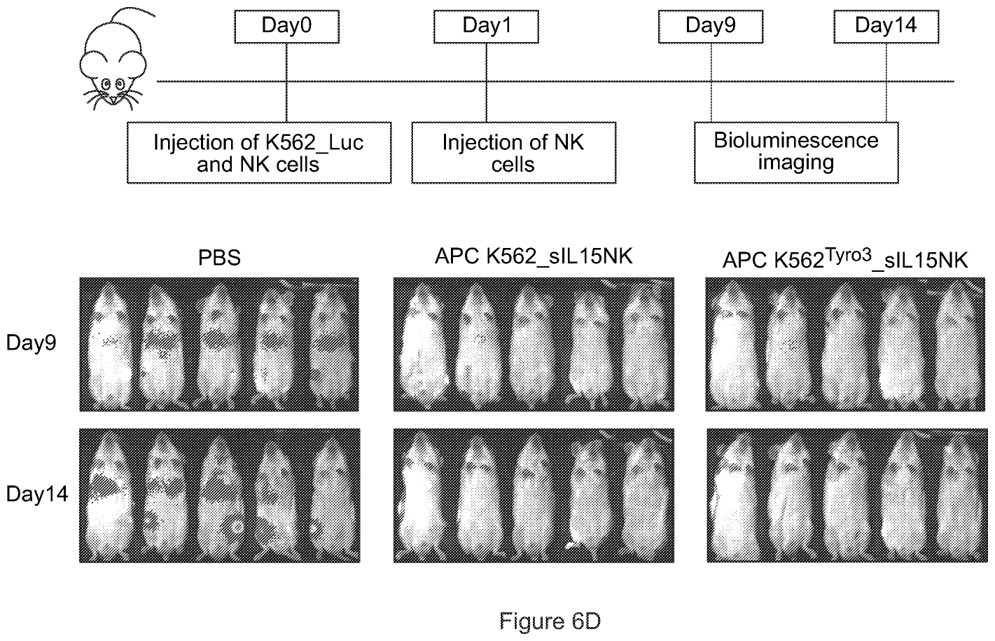

Since acquisition of Tyro3 by NK cells from APC K562 cells expressing Tyro3 enhances NK cell ex vivo expansion, NK cells function was assessed both in vivo and in vitro. Primary human NK cells were incubated with inactivated APC K562 or APC K562^{Tyro3} in the presence of IL-2 (50 IU/ml) for 7 days and followed by detecting the expression of surface markers on these NK cells. Regarding activating surface markers of NK cells, the expression of CD16, DNAM-1, NKG2D, NKp30, and NKp46 did not show any differences between APC K562^{Tyro3} and APC K562 expanded NK cells. NK cells expanded by APC K562^{Tyro3} expressed higher levels of CD25 and CD62L but lower levels of CD69 and NKp44 than NK cells expanded by APC K562 (FIG. 6A). The function of expanded NK cells was also assessed via a CD107a degradation assay and ⁵¹Cr cytotoxicity assessment, and data showed that NK cells expanded with APC K562^{Tyro3} and APC K562 showed similar levels of CD107a degranulation (FIG. 6B) and cytotoxicity (FIG. 6C).

Next, the ability of NK cells expanded by APC K562^{Tyro3} to control tumors in vivo was investigated by engrafting NOD-SCID-IL2Rγ^{-/-} (NSG) mice with luciferase-expressing K562 cells (K562_Luc), followed by treating them with or without soluble (s) IL-15 expressing NK cells expanded by APC K562 (APC K562_sIL15 NK cells) or APC K562^{Tyro3} (APC K562^{Tyro3}_sIL15 NK cells). APC K562^{Tyro3}_sIL15 NK cells were found to significantly suppress tumor burden at a level similar to that of APC K562_sIL15 NK cells, which was assessed by whole-body bioluminescence imaging (FIG. 6D). Thus, APC K562^{Tyro3} sIL15 NK cells display similar functions as APC K562_sIL15 NK cells in vitro and in vivo.

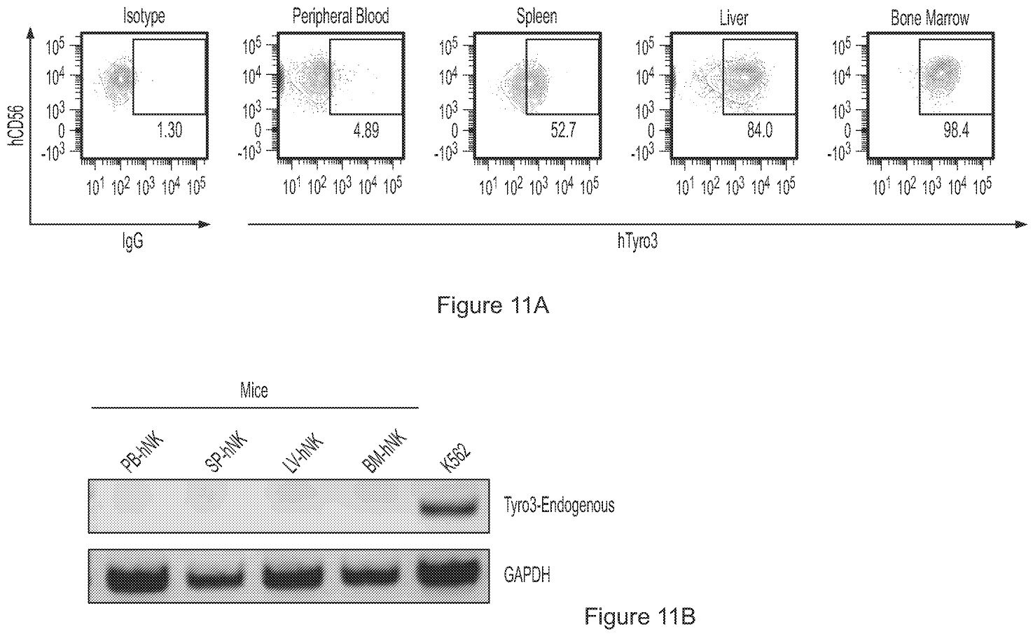

Finally, the occurrence of Tyro3 trogocytosis from tumor cells to NK cells was studied in vivo. For this purpose, NSG mice were engrafted with K562_Luc cells for 14 days, followed by treatment with sIL15 NK cells, and Tyro3 expression in human NK cells was detected in different organs or tissues of mice 2 days post-treatment. Tyro3 was found to be highly expressed in human NK cells from the bone marrow and liver, moderately in the spleen, but barely in the peripheral blood of mice (FIG. 11A). To assess if Tyro3 in these human NK cells was due to endogenous expression of the Tyro3 gene, human NK cells were sorted from different organs or tissues of mice, followed by detection of Tyro3 at the mRNA level by RT-PCR. Our data showed that the endogenous expression of Tyro3 in these human NK cells was undetectable (FIG. 11B), indicating that Tyro3 on those NK cells are acquired from K562_Luc cells in vivo via trogocytosis.

### OTHER EMBODIMENTS

It is to be understood that while the invention has been described in conjunction with the detailed description thereof, the foregoing description is intended to illustrate and not limit the scope of the invention.

1. Modified K562 myeloid leukemia cells (Tyro3⁺ K562 cells) expressing human Tyro3 polypeptide comprising an amino acid sequence that is at least 95% identical to SEQ ID NO:2, and wherein the Tyro3⁺ K562 cells express membrane bound interleukin 21 (IL-21) and 4-1 BB ligand (4-1BBL).

2. The Tyro3⁺ K562 cells of claim 1, wherein the Tyro3⁺ K562 cells enhance the expansion of human natural killer (NK) cells that contact the K562 cells by at least about 10% compared to K562 cells that do not express exogenous human Tyro3 (Tyro3⁻ K562 cells).

3. The Tyro3⁺ K562 cells of claim 1, wherein the human Tyro3 expression is under the control of a strong promoter.

4. The Tyro3⁺ K562 cells of claim 3, wherein the strong promoter is a retroviral promoter.

5. A method of expanding a population of human NK cells, comprising co-culturing the population of NK cells with K562 myeloid leukemia cells, wherein the K562 cells (Tyro3⁺ K562 cells) are modified to exogenously express human Tyro3 polypeptide, which polypeptide comprising an amino acid sequence that is at least 95% identical to SEQ ID NO:2, wherein the human Tyro3 gene expression is under the control of a constitutive promoter, and wherein the K562 cell expresses membrane bound interleukin 21 (IL-21) and 4-1 BB ligand (4-1BBL).

6. The method of claim 5, wherein Tyro3 is transferred from the Tyro3⁺ K562 cells to the human NK cells by trogocytosis to obtain Tyro3⁺ NK cells.

7. The method of claim 5, wherein the human NK cells are expanded in the presence of interleukin 2 (IL-2), wherein the IL-2 is present at a concentration of about 0 IU/ml to about 5000 IU/ml, about 50 IU/ml to about 2000 IU/ml, or about 150 IU/ml to about 900 IU/ml.

8.-12. (canceled)

13. The method of claim 5, wherein the expanded NK cells are activated NK cells.

14. The method of claim 5, wherein the NK cells are engineered NK cells, wherein the engineered NK cells express a chimeric antigen receptor (CAR).

15. (canceled)

16. The method of claim 14, wherein the chimeric antigen receptor (CAR) expressed in the engineered NK cell is selected from the group consisting of CD19, CD20, CD22, CD30, CD33, CD38, CD70, CD123, Kappa, NKG2D ligands, ROR1; CAR targets for solid tumors such as B7H3 CD44 v6/v7, CD171, CEA, EGFRvIII, EGP2, EGP40, EphA2, ErbB2(HER2), ErbB receptor family, ErbB3/4, HLA-A1/MAGE1, HLA-A2/NY-ESO-1, FR-α, FAP, FAR, GD2, GD3, HMW-MAA, IL11Rα, IL13Rα2, Lewis Y, Mesothelin, Muel, PSCA, HPB, PSMA, TAG72, and VEGFR-2.

17. The method of claim 5, wherein the expanded NK cells are Programmed death-ligand 1 (PD-L1)⁺ cells.

18. The method of claim 5, wherein the expanded NK cells have at least one of:
(a) higher levels of mRNA and/or protein of one or more of the following markers: phosphorylated-signal transducer and activator of transcription 3 (p-STAT3), phosphorylated-NF-κB p65 (p-P65), phospho-Akt (p-AKT) and phospho-extracellular signal-related kinase (p-ERK), Interferon-gamma (IFNγ), Cluster of differentiation 107a (CD107a), CD25, CD69, Killer cell lectin-like receptor subfamily G member 1 (KLRG1), when compared to reference levels of the corresponding mRNA and/or protein in a control; and
(b) higher levels of activity of one or more of the following markers: p-STAT3, p-P65, p-AKT, p-ERK, IFNγ, CD107a, CD25, CD69, and KLRG1, when compared to reference levels of the corresponding activity in a control; or
wherein the expanded NK cells have at least one of:
(a) higher levels of mRNA and/or protein of one or more of the following markers: phosphorylated-signal transducer and activator of transcription 5 (p-STAT5), phospho-Akt (p-AKT) and phospho-extracellular signal-related kinase (p-ERK), Interferon-gamma (IFNγ), Cluster of differentiation 107a (CD107a), CD25, CD69, Killer cell lectin-like receptor subfamily G member 1 (KLRG1), when compared to reference levels of the corresponding mRNA and/or protein in a control; and
(b) higher levels of activity of one or more of the following markers: p-STAT3, p-P65, p-AKT, p-ERK, IFNγ, CD107a, CD25, CD69, and KLRG1, when compared to reference levels of the corresponding activity in a control.

19. (canceled)

20. A composition comprising the population of expanded NK cells produced by the method claim 5.

21. A composition comprising expanded NK cells, wherein at least 20% of the NK cells in the population are Tyro3⁺ NK cells.

22. A method of treating cancer in a subject in need thereof, comprising administering to the subject a therapeutically effective amount of a composition of claim 20, thereby treating cancer in the subject.

23. The method of claim 22, wherein the cancer is selected from a group consisting of lung cancer, breast cancer, ewing sarcoma, central nervous system neoplasm, skin cancer, head and neck cancer, ovarian cancer, colon cancer, anal cancer, stomach cancer, gastrointestinal cancer, fallopian tube cancer, endometrial cancer, cervical cancer, vaginal cancer, vulvar cancer, esophageal cancer, endocrine cancer, thyroid cancer, parathyroid cancer, adrenal cancer, soft tissue sarcoma, urethral cancer, penile cancer, testicular cancer, brain stem glioma, pituitary cancer, adrenocortical cancer, gallbladder cancer, multiple myeloma, cholangiocarcinoma, fibrosarcoma, lymphoma, liver cancer, kidney cancer, bone cancer, bladder cancer, colorectal cancer, endometrial cancer, renal cell cancer, pancreatic cancer, prostate cancer, thyroid cancer, mesothelioma, neuroblastoma, retinoblastoma, melanoma, rhabdomyosarcoma, leukemia and lymphoma.

24. A method of treating a viral infection in a subject in need thereof, comprising administering to the subject a therapeutically effective amount of a composition of claim 20, thereby treating the viral infection in the subject.

25. The method of claim 24, wherein the viral infection is caused by human immunodeficiency virus (HIV), Epstein-Barr virus (EBV), herpes simplex virus (HSV), cytomegalovirus (CMV), varicella-zoster virus (VZV), hepatitis B virus (HBV) or hepatitis C virus (HCV), and coronavirus.

26. A method of suppressing the proliferation of tumor cells comprising contacting the tumor cells with a therapeutically effective amount of a composition of claim 20.

27. The method of claim 26, wherein said tumor cells are primary ductal carcinoma cells, glioblastoma cells, leukemia cells, acute T cell leukemia cells, chronic myeloid lymphoma (CML) cells, acute myelogenous leukemia cells, chronic myelogenous leukemia (CML) cells, lung carcinoma cells, colon adenocarcinoma cells, histiocytic lymphoma cells, multiple myeloma cells, colorectal carcinoma cells, colorectal adenocarcinoma cells, prostate cancer cells, or retinoblastoma cells.

28.-30. (canceled)


SEQUENCE LISTING (27 sequences)

SEQ ID NO: 1 - DNA, 2673 bp, Artificial Sequence
  source: /note=“Description of Artificial Sequence Synthetic polynucleotide”
atggcgctgaggcggagcatggggcggccggggctcccgccgctgccgctgccgccgccaccgcggctcgggctgctgctggcggctctggcttctctgctgctcccggagtccgccgccgcaggtctgaagctcatgggagccccggtgaagctgacagtgtctcaggggcagccggtgaagctcaactgcagtgtggaggggatggaggagcctgacatccagtgggtgaaggatggggctgtggtccagaacttggaccagttgtacatcccagtcagcgagcagcactggatcggcttcctcagcctgaagtcagtggagcgctctgacgccggccggtactggtgccaggtggaggatgggggtgaaaccgagatctcccagccagtgtggctcacggtagaaggtgtgccatttttcacagtggagccaaaagatctggcagtgccacccaatgcccctttccaactgtcttgtgaggctgtgggtccccctgaacctgttaccattgtctggtggagaggaactacgaagatcgggggacccgctccctctccatctgttttaaatgtaacaggggtgacccagagcaccatgttttcctgtgaagctcacaacctaaaaggcctggcctcttctcgcacagccactgttcaccttcaagcactgcctgcagcccccttcaacatcaccgtgacaaagctttccagcagcaacgctagtgtggcctggatgccaggtgctgatggccgagctctgctacagtcctgtacagttcaggtgacacaggccccaggaggctgggaagtcctggctgttgtggtccctgtgcccccctttacctgcctgctccgggacctggtgcctgccaccaactacagcctcagggtgcgctgtgccaatgccttggggccctctccctatgctgactgggtgccctttcagaccaagggtctagccccagccagcgctccccaaaacctccatgccatccgcacagattcaggcctcatcttggagtgggaagaagtgatccccgaggcccctttggaaggccccctgggaccctacaaactgtcctgggttcaagacaatggaacccaggatgagctgacagtggaggggaccagggccaatttgacaggctgggatccccaaaaggacctgatcgtacgtgtgtgcgtctccaatgcagttggctgtggaccctggagtcagccactggtggtctcttctcatgaccgtgcaggccagcagggccctcctcacagccgcacatcctgggtacctgtggtccttggtgtgctaacggccctggtgacggctgctgccctggccctcatcctgcttcgaaagagacggaaagagacgcggtttgggcaagcctttgacagtgtcatggcccggggagagccagccgttcacttccgggcagcccggtccttcaatcgagaaaggcccgagcgcatcgaggccacattggacagcttgggcatcagcgatgaactaaaggaaaaactggaggatgtgctcatcccagagcagcagttcaccctgggccggatgttgggcaaaggagagtttggttcagtgcgggaggcccagctgaagcaagaggatggctcctttgtgaaagtggctgtgaagatgctgaaagctgacatcattgcctcaagcgacattgaagagttcctcagggaagcagcttgcatgaaggagtttgaccatccacacgtggccaaacttgttggggtaagcctccggagcagggctaaaggccgtctccccatccccatggtcatcttgcccttcatgaagcatggggacctgcatgccttcctgctcgcctcccggattggggagaacccctttaacctacccctccagaccctgatccggttcatggtggacattgcctgcggcatggagtacctgagctctcggaacttcatccaccgagacctggctgctcggaattgcatgctggcagaggacatgacagtgtgtgtggctgacttcggactctcccggaagatctacagtggggactactatcgtcaaggctgtgcctccaaactgcctgtcaagtggctggccctggagagcctggccgacaacctgtatactgtgcagagtgacgtgtgggcgttcggggtgaccatgtgggagatcatgacacgtgggcagacgccatatgctggcatcgaaaacgctgagatttacaactacctcattggcgggaaccgcctgaaacagcctccggagtgtatggaggacgtgtatgatctcatgtaccagtgctggagtgctgaccccaagcagcgcccgagctttacttgtctgcgaatggaactggagaacatcttgggccagctgtctgtgctatctgccagccaggaccccttatacatcaacatcgagagagctgaggagcccactgcgggaggcagcctggagctacctggcagggatcagccctacagtggggctggggatggcagtggcatgggggcagtgggtggcactcccagtgactgtcggtacatactcacccccggagggctggctgagcagccagggcaggcagagcaccagccagagagtcccctcaatgagacacagaggcttttgctgctgcagcaagggctactgccacacagtagctgttga

SEQ ID NO: 2 - protein, 890 aa, Artificial Sequence
  source: /note=“Description of Artificial Sequence Synthetic polypeptide”
MALRRSMGRPGLPPLPLPPPPRLGLLLAALASLLLPESAAAGLKLMGAPVKLTVSQGQPVKLNCSVEGMEEPDIQWVKDGAVVQNLDQLYIPVSEQHWIGFLSLKSVERSDAGRYWCQVEDGGETEISQPVWLTVEGVPFFTVEPKDLAVPPNAPFQLSCEAVGPPEPVTIVWWRGTTKIGGPAPSPSVLNVTGVTQSTMFSCEAHNLKGLASSRTATVHLQALPAAPFNITVTKLSSSNASVAWMPGADGRALLQSCTVQVTQAPGGWEVLAVVVPVPPFTCLLRDLVPATNYSLRVRCANALGPSPYADWVPFQTKGLAPASAPQNLHAIRTDSGLILEWEEVIPEAPLEGPLGPYKLSWVQDNGTQDELTVEGTRANLTGWDPQKDLIVRVCVSNAVGCGPWSQPLVVSSHDRAGQQGPPHSRTSWVPVVLGVLTALVTAAALALILLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSC

SEQ ID NO: 3 - DNA, 3414 bp, Artificial Sequence
  source: /note=“Description of Artificial Sequence Synthetic polynucleotide”
atggcgctgaggcggagcatggggcggccggggctcccgccgctgccgctgccgccgccaccgcggctcgggctgctgctggcggctctggcttctctgctgctcccggagtccgccgccgcaggtctgaagctcatgggagccccggtgaagctgacagtgtctcaggggcagccggtgaagctcaactgcagtgtggaggggatggaggagcctgacatccagtgggtgaaggatggggctgtggtccagaacttggaccagttgtacatcccagtcagcgagcagcactggatcggcttcctcagcctgaagtcagtggagcgctctgacgccggccggtactggtgccaggtggaggatgggggtgaaaccgagatctcccagccagtgtggctcacggtagaaggtgtgccatttttcacagtggagccaaaagatctggcagtgccacccaatgcccctttccaactgtcttgtgaggctgtgggtccccctgaacctgttaccattgtctggtggagaggaactacgaagatcgggggacccgctccctctccatctgttttaaatgtaacaggggtgacccagagcaccatgttttcctgtgaagctcacaacctaaaaggcctggcctcttctcgcacagccactgttcaccttcaagcactgcctgcagcccccttcaacatcaccgtgacaaagctttccagcagcaacgctagtgtggcctggatgccaggtgctgatggccgagctctgctacagtcctgtacagttcaggtgacacaggccccaggaggctgggaagtcctggctgttgtggtccctgtgcccccctttacctgcctgctccgggacctggtgcctgccaccaactacagcctcagggtgcgctgtgccaatgccttggggccctctccctatgctgactgggtgccctttcagaccaagggtctagccccagccagcgctccccaaaacctccatgccatccgcacagattcaggcctcatcttggagtgggaagaagtgatccccgaggcccctttggaaggccccctgggaccctacaaactgtcctgggttcaagacaatggaacccaggatgagctgacagtggaggggaccagggccaatttgacaggctgggatccccaaaaggacctgatcgtacgtgtgtgcgtctccaatgcagttggctgtggaccctggagtcagccactggtggtctcttctcatgaccgtgcaggccagcagggccctcctcacagccgcacatcctgggtacctgtggtccttggtgtgctaacggccctggtgacggctgctgccctggccctcatcctgcttcgaaagagacggaaagagacgcggtttgggcaagcctttgacagtgtcatggcccggggagagccagccgttcacttccgggcagcccggtccttcaatcgagaaaggcccgagcgcatcgaggccacattggacagcttgggcatcagcgatgaactaaaggaaaaactggaggatgtgctcatcccagagcagcagttcaccctgggccggatgttgggcaaaggagagtttggttcagtgcgggaggcccagctgaagcaagaggatggctcctttgtgaaagtggctgtgaagatgctgaaagctgacatcattgcctcaagcgacattgaagagttcctcagggaagcagcttgcatgaaggagtttgaccatccacacgtggccaaacttgttggggtaagcctccggagcagggctaaaggccgtctccccatccccatggtcatcttgcccttcatgaagcatggggacctgcatgccttcctgctcgcctcccggattggggagaacccctttaacctacccctccagaccctgatccggttcatggtggacattgcctgcggcatggagtacctgagctctcggaacttcatccaccgagacctggctgctcggaattgcatgctggcagaggacatgacagtgtgtgtggctgacttcggactctcccggaagatctacagtggggactactatcgtcaaggctgtgcctccaaactgcctgtcaagtggctggccctggagagcctggccgacaacctgtatactgtgcagagtgacgtgtgggcgttcggggtgaccatgtgggagatcatgacacgtgggcagacgccatatgctggcatcgaaaacgctgagatttacaactacctcattggcgggaaccgcctgaaacagcctccggagtgtatggaggacgtgtatgatctcatgtaccagtgctggagtgctgaccccaagcagcgcccgagctttacttgtctgcgaatggaactggagaacatcttgggccagctgtctgtgctatctgccagccaggaccccttatacatcaacatcgagagagctgaggagcccactgcgggaggcagcctggagctacctggcagggatcagccctacagtggggctggggatggcagtggcatgggggcagtgggtggcactcccagtgactgtcggtacatactcacccccggagggctggctgagcagccagggcaggcagagcaccagccagagagtcccctcaatgagacacagaggcttttgctgctgcagcaagggctactgccacacagtagctgtggaggaggaggaagtgcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 4 - protein, 1137 aa, Artificial Sequence
  source: /note=“Description of Artificial Sequence Synthetic polypeptide”
MALRRSMGRPGLPPLPLPPPPRLGLLLAALASLLLPESAAAGLKLMGAPVKLTVSQGQPVKLNCSVEGMEEPDIQWVKDGAVVQNLDQLYIPVSEQHWIGFLSLKSVERSDAGRYWCQVEDGGETEISQPVWLTVEGVPFFTVEPKDLAVPPNAPFQLSCEAVGPPEPVTIVWWRGTTKIGGPAPSPSVLNVTGVTQSTMFSCEAHNLKGLASSRTATVHLQALPAAPFNITVTKLSSSNASVAWMPGADGRALLQSCTVQVTQAPGGWEVLAVVVPVPPFTCLLRDLVPATNYSLRVRCANALGPSPYADWVPFQTKGLAPASAPQNLHAIRTDSGLILEWEEVIPEAPLEGPLGPYKLSWVQDNGTQDELTVEGTRANLTGWDPQKDLIVRVCVSNAVGCGPWSQPLVVSSHDRAGQQGPPHSRTSWVPVVLGVLTALVTAAALALILLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSCGGGGSAAAMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 5 - DNA, 1287 bp, Artificial Sequence
  source: /note=“Description of Artificial Sequence Synthetic polynucleotide”
atggcgctgaggcggagcatggggcggccggggctcccgccgctgccgctgccgccgccaccgcggctcgggctgctgctggcggctctggcttctctgctgctcccggagtccgccgccgcaggtctgaagctcatgggagccccggtgaagctgacagtgtctcaggggcagccggtgaagctcaactgcagtgtggaggggatggaggagcctgacatccagtgggtgaaggatggggctgtggtccagaacttggaccagttgtacatcccagtcagcgagcagcactggatcggcttcctcagcctgaagtcagtggagcgctctgacgccggccggtactggtgccaggtggaggatgggggtgaaaccgagatctcccagccagtgtggctcacggtagaaggtgtgccatttttcacagtggagccaaaagatctggcagtgccacccaatgcccctttccaactgtcttgtgaggctgtgggtccccctgaacctgttaccattgtctggtggagaggaactacgaagatcgggggacccgctccctctccatctgttttaaatgtaacaggggtgacccagagcaccatgttttcctgtgaagctcacaacctaaaaggcctggcctcttctcgcacagccactgttcaccttcaagcactgcctgcagcccccttcaacatcaccgtgacaaagctttccagcagcaacgctagtgtggcctggatgccaggtgctgatggccgagctctgctacagtcctgtacagttcaggtgacacaggccccaggaggctgggaagtcctggctgttgtggtccctgtgcccccctttacctgcctgctccgggacctggtgcctgccaccaactacagcctcagggtgcgctgtgccaatgccttggggccctctccctatgctgactgggtgccctttcagaccaagggtctagccccagccagcgctccccaaaacctccatgccatccgcacagattcaggcctcatcttggagtgggaagaagtgatccccgaggcccctttggaaggccccctgggaccctacaaactgtcctgggttcaagacaatggaacccaggatgagctgacagtggaggggaccagggccaatttgacaggctgggatccccaaaaggacctgatcgtacgtgtgtgcgtctccaatgcagttggctgtggaccctggagtcagccactggtggtctcttctcatgaccgtgcaggccagcagggccctcctcacagccgcacatcctgg

SEQ ID NO: 6 - protein, 429 aa, Artificial Sequence
  source: /note=“Description of Artificial Sequence Synthetic polypeptide”
MALRRSMGRPGLPPLPLPPPPRLGLLLAALASLLLPESAAAGLKLMGAPVKLTVSQGQPVKLNCSVEGMEEPDIQWVKDGAVVQNLDQLYIPVSEQHWIGFLSLKSVERSDAGRYWCQVEDGGETEISQPVWLTVEGVPFFTVEPKDLAVPPNAPFQLSCEAVGPPEPVTIVWWRGTTKIGGPAPSPSVLNVTGVTQSTMFSCEAHNLKGLASSRTATVHLQALPAAPFNITVTKLSSSNASVAWMPGADGRALLQSCTVQVTQAPGGWEVLAVVVPVPPFTCLLRDLVPATNYSLRVRCANALGPSPYADWVPFQTKGLAPASAPQNLHAIRTDSGLILEWEEVIPEAPLEGPLGPYKLSWVQDNGTQDELTVEGTRANLTGWDPQKDLIVRVCVSNAVGCGPWSQPLVVSSHDRAGQQGPPHSRTSW

SEQ ID NO: 7 - DNA, 147 bp, Artificial Sequence
  source: /note=“Description of Artificial Sequence Synthetic polynucleotide”
gctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 8 - protein, 49 aa, Artificial Sequence
  source: /note=“Description of Artificial Sequence Synthetic polypeptide”
AVGQDTQEVIVVPHSLPFKVVVISAILALVVLTIISLIILIMLWQKKPR

SEQ ID NO: 9 - DNA, 24 bp, Artificial Sequence
  source: /note=“Description of Artificial Sequence Synthetic primer”
ctaattattcggtaactgacttga

SEQ ID NO: 10 - DNA, 21 bp, Artificial Sequence
  source: /note=“Description of Artificial Sequence Synthetic primer”
acagttcagccatcacttgga

SEQ ID NO: 11 - DNA, 21 bp, Artificial Sequence
  source: /note=“Description of Artificial Sequence Synthetic primer”
tgggggacccagagattaaaa

SEQ ID NO: 12 - DNA, 22 bp, Artificial Sequence
  source: /note=“Description of Artificial Sequence Synthetic primer”
tttcgtccataggagacaatgc

SEQ ID NO: 13 - DNA, 21 bp, Artificial Sequence
  source: /note=“Description of Artificial Sequence Synthetic primer”
cgcctacctcaggcttatctc

SEQ ID NO: 14 - DNA, 19 bp, Artificial Sequence
  source: /note=“Description of Artificial Sequence Synthetic primer”
cctcgacagtcaggcagtc

SEQ ID NO: 15 - DNA, 20 bp, Artificial Sequence
  source: /note=“Description of Artificial Sequence Synthetic primer”
tgcatggccgttcttagttg

SEQ ID NO: 16 - DNA, 23 bp, Artificial Sequence
  source: /note=“Description of Artificial Sequence Synthetic primer”
agttagcatgccagagtctcgtt

SEQ ID NO: 17 - DNA, 22 bp, Artificial Sequence
  source: /note=“Description of Artificial Sequence Synthetic primer”
cagtgactgtcggtacatactc

SEQ ID NO: 18 - DNA, 20 bp, Artificial Sequence
  source: /note=“Description of Artificial Sequence Synthetic primer”
catcaagccaaggcctaaac

SEQ ID NO: 19 - DNA, 22 bp, Artificial Sequence
  source: /note=“Description of Artificial Sequence Synthetic primer”
cagtgactgtcggtacatactc

SEQ ID NO: 20 - DNA, 20 bp, Artificial Sequence
  source: /note=“Description of Artificial Sequence Synthetic primer”
ctgaacttgtggccgtttac

SEQ ID NO: 21 - DNA, 26 bp, Artificial Sequence
  source: /note=“Description of Artificial Sequence Synthetic primer”
atggggaaggtgaaggtcggagtcaa

SEQ ID NO: 22 - DNA, 24 bp, Artificial Sequence
  source: /note=“Description of Artificial Sequence Synthetic primer”
cggaggggccatccacagtcttct

SEQ ID NO: 23 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 24 - DNA, 716 bp, Homo sapiens
ctcagtcaagctgaagtgaaaacgagaccaaggtctagctctactgttggtacttatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaaggtatctaccttaagtttcatttgattttctgctttatctttacctatccagatttgcttcttagttactcacggtatactatttccacagatgattcatcagcatctgtcctctagaacacacggaagtgaagattcctgaggatctaacttgcagttggacactatgttacatactctaatatagtagtgaaagtcatttctttgtattccaagtggaggag

SEQ ID NO: 25 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 26 - DNA, 1634 bp, Homo sapiens
agtctctcgtcatggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataacgtccagcctgggtgcagcccacctggacagagtccgaatcctactccatccttcatggagacccctggtgctgggtccctgctgctttctctacctcaaggggcttggcaggggtccctgctgctgacctccccttgaggaccctcctcacccactccttccccaagttggaccttgatatttattctgagcctgagctcagataatatattatatatattatatatatatatatatttctatttaaagaggatcctgagtttgtgaatggacttttttagaggagttgttttgggggggggggggtcttcgacattgccgaggctggtcttgaactcctggacttagacgatcctcctgcctcagcctcccaagcaactgggattcatcctttctattaattcattgtacttatttgcttatttgtgtgtattgagcatctgtaatgtgccagcattgtgcccaggctagggggctatagaaacatctagaaatagactgaaagaaaatctgagttatggtaatacgtgaggaatttaaagactcatccccagcctccacctcctgtgtgatacttgggggctagcttttttctttctttcttttttttgagatggtcttgttctgtcaaccaggctagaatgcagcggtgcaatcatgagtcaatgcagcctccagcctcgacctcccgaggctcaggtgatcctcccatctcagcctctcgagtagctgggaccacagttgtgtgccaccacacttggctaactttttaatttttttgcggagacggtattgctatgttgccaaggttgtttacatgccagtacaatttataataaacactcatttttcctccc

SEQ ID NO: 27 - protein, 5 aa, Artificial Sequence
  source: /note=“Description of Artificial Sequence Synthetic peptide”
GGGGS